(19) United States
(12) Patent Application Publication
(10) Pub. No.: US 2023/0277518 A1
(43) Pub. Date: Sep. 7, 2023

(54) NK RECEPTOR ANTAGONISTS FOR CANCER PATIENTS

(71) Applicants: Acer Therapeutics Inc., Newton, MA (US); University of Washington, Seattle, WA (US)

(72) Inventors: Robert A. STEINER, Bainbridge lsland, WA (US); D. Christopher SCHELLING, Bend, OR (US); Susan D. REED, Bainbridge Island, WA (US); Victor M. NAVARRO, Boston, MA (US)

(21) Appl. No.: 18/017,811

(22) PCT Filed: Jul. 30, 2021
(86) PCT No.: PCT/US2021/044016

§ 371 Date: Jan. 24, 2023

Related U.S. Application Data
(60) Provisional application No. 63/134,542, filed on Jan. 6, 2021.
(60) Provisional application No. 63/059,086, filed on Jul. 30, 2020.

(51) Int. Cl. A61K 31/4545; A61K 31/138; A61K 38/09; A61P 35/00; A61P 13/08

(52) U.S. Cl. CPC A61K 31/4545; A61K 31/138; A61K 38/09; A61P 35/00; A61P 13/08

(57) ABSTRACT

The present disclosure relates generally to a method of blocking, attenuating, or limiting the development of one or more vasomotor symptoms (VMS) in a patient who has cancer, has had cancer, or has an increased risk for cancer by administering a NK antagonist.

### CROSS REFERENCE TO RELATED APPLICATIONS

This application claims the benefit of priority under 35 U.S.C. § 119(e) to U.S. Provisional Application No. 63/059,086, filed Jul. 30, 2020, and to U.S. Provisional Application No. 63/134,542, filed Jan. 6, 2021, which are incorporated herein by reference in their entireties.

### FIELD

Provided herein are methods for blocking, attenuating, or limiting the development of one or more vasomotor symptoms (VMS) in a patient who has cancer, has had cancer, or has an increased risk for cancer by administering a NK receptor antagonist.

### BACKGROUND

Cancer is a genetic disease in which a cell's genes are atypical, either arising from inherited genetic predisposition or the accumulation of DNA damage over a person's lifetime due to environmental exposure or unresolved errors occurring during cell division. These genetically aberrant cells may not respond to normal cell lifecycle signals, such as when to stop replicating or when to die. In these cases, the abnormal cells may reproduce unchecked and potentially spread to surrounding tissues and other organs in the body, negatively impacting neighboring cells ability to properly function.

In the course of attempting to prevent or treat several cancers of the reproductive system, the removal of affected organs is a common surgical intervention. These organs are removed in order to excise malignant cells, and to remove the primary source of endogenous sex hormones, which are linked to the growth and proliferation of certain hormone-dependent cancers. However, the loss of these sex-hormone producing organs may cause unintended side-effects. Vasomotor symptoms (VMS), such as hot flashes and night sweats, are a common symptom of patients who have undergone a medical or surgical intervention to treat cancer, which has affected the balance of their sex-hormones.

Vasomotor symptoms (VMS, hot flashes) are common in cancer patients treated with hormone-deprivation therapy, or in patients who have undergone surgery such as removal of ovaries or the prostate gland. About 60%-70% of women treated with tamoxifen experience increased frequency and/or duration of VMS and about 80% of men treated with leuprolide experience increased frequency and/or duration of VMS. Hormone deprivation (e.g., post-oophorectomy, post-prostectomy, or from treatment with tamoxifen or leuprolide) can result in KNDy neuron hypertrophy and increased expression of neurokinin B (NKB). The increased concentrations of NKB trigger VMS. When NK antagonists are administered, they can lower the production of NKB thereby alleviating VMS.

### SUMMARY

It has now been found that in cancer patients who are being treated with hormone deprivation therapy, or having medical or surgical procedures, the administration of NK antagonists blocks, attenuates, or limits the development of one or more vasomotor symptoms. Unexpectedly it was found that twice daily dosing of a NK antagonist decreased VMS more than once daily dosing of the NK antagonist. In other words, splitting the total daily dose of the NK antagonist confers superior therapeutic benefit compared to a single administration of the total daily dose of the antagonist.

Provided herein is a method of preventing, including blocking, attenuating, or limiting, the development of one or more vasomotor symptoms (VMS) in a patient wherein the patient will be undergoing hormone deprivation therapy, a medical and/or surgical procedure that may cause VMS, comprising administering an effective amount of a neurokinin receptor (NK) antagonist, for a time period prior to, and optionally concurrently with, the hormone deprivation therapy, a medical and/or surgical procedure. In certain embodiments, the patient has cancer, has had cancer, or is at an increased risk for cancer. In certain embodiments, the administration of the NK antagonist is administered for a period of time prior to the hormone deprivation therapy, and/or medical or surgical procedure. In certain embodiments one or more NK antagonist is administered. In certain embodiments, the NK receptor antagonist is a NK3 antagonist. In certain embodiments, provided is a method of preventing hypertrophy kisspeptin/neurokinin B/dynorphin (KNDy) neurons in a patient in need thereof by administering to said patient an effective amount of a NK antagonist.

In certain embodiments, the NK antagonist is osanetant or a stereoisomer, mixture of stereoisomers, prodrug, pharmaceutically acceptable salt, hydrate, solvate, acid salt hydrate, N-oxide or isomorphic crystalline form thereof. In certain embodiments, the effective dose of the NK antagonist, or osanetant, the effective dose for the induced VMS from the hormone deprivation therapy, a medical and/or surgical procedure will be lower than the effective dose from treating VMS in post-menopausal women. In certain embodiments, the patients continues the NK antagonist therapy.

### DESCRIPTION OF DRAWINGS

FIG. 1 demonstrates that leuprolide treatment in a virtual patient decreases testosterone to castration levels of <50 ng/dL. Osanetant co-administration does not increase the testosterone concentrations.

FIG. 2A demonstrates that in a post-menopausal virtual patient, treatment with tamoxifen may not cause large increases in estradiol. FIG. 2B demonstrates that in a post-menopausal virtual patient, treatment with tamoxifen may result in changes in GnRH and NKB.

FIG. 3 shows a comparison of two dosing regimens, once daily dosing (QD, left) and twice daily dosing (BID, right) of the same total daily dose. BID dosing decreases VMS frequency compared to QD dosing.

FIG. 4 demonstrates that in a female virtual patient being treated with tamoxifen, co-administration of osanetant, 150 mg BID reduces both frequency and severity of VMS.

FIG. 5 demonstrates that in a male virtual patient, leuprolide treatment increases VMS. Co-administration of osanetant with leuprolide reduces VMS to near 0 in this virtual patient.

FIG. 6 shows a comparison of model simulation to clinical data for tamoxifen. Induced VMS are often measured as a combined score. In women, tamoxifen therapy increases VMS.

FIG. 7 shows a comparison of model simulation to clinical data for leuprolide. Induced VMS are often measured as a combined score. In men, VMS increases over time.

FIG. 8A shows a comparison of model simulation to a reported study for osanetant. A two-compartment pharmacokinetic model was fit to a single-dose study for osanetant which was conducted by Sanofi. Certain data from the Sanofi study is shown in FIG. 8B.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

Unless defined otherwise, all technical and scientific terms used herein have the same meaning as is commonly understood by one of ordinary skill in the art. As used herein, the below terms have the following meanings unless specified otherwise. Any methods, devices and materials similar or equivalent to those described herein can be used in the practice of the compositions and methods described herein. The following definitions are provided to facilitate understanding of certain terms used frequently herein and are not meant to limit the scope of the present disclosure. All references referred to herein are incorporated by reference in their entirety.

The term “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, references to “the agent” includes a plurality of such agents.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X.”

“Pharmaceutically acceptable” or “physiologically acceptable” refer to compounds, salts, compositions, dosage forms and other materials which are useful in preparing a pharmaceutical composition that is suitable for human or veterinary pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposeable mirror images of one another.

A “prodrug” is any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate and benzoate derivatives), amides, guanidines, carbamates (e.g., N,N-dimethylaminocarbonyl) of hydroxy functional groups in compounds described herein and the like. Preparation, selection and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

As used herein, the term “solvate” refers to a complex formed by combining a compound and a solvent.

As used herein, the term “hydrate” refers to a complex formed by combining a compound and water (i.e., a solvate when the solvent is water).

As used herein, the term “acid salt hydrate” refers to a complex formed by combining an acid salt compound with water.

As used herein, the term “N-oxide” refers to an oxidized tertiary or pyridinyl amine moiety.

As used herein, the term “isomorphic crystalline form” refers to two or more crystalline forms that have the same space group, unit-cell dimensions, and types and positions of atoms, with the exception of a replacement of one or more atoms in one isomorphic crystalline form with a different atom in its counterpart isomorphic crystalline form.

As used herein, the term “administration” refers to introducing an agent into a patient. For example, a therapeutic amount can be administered to the patient, which can be determined by the treating physician, medical professional, or the like. In some embodiments, a therapeutic amount is administered orally. In some embodiments, a therapeutic amount is administered intranasally. In some embodiments, a therapeutic amount is administered subcutaneously. In some embodiments, a therapeutic amount is administered transdermally. In some embodiments, a therapeutic amount is administered intravenously. In some embodiments, a therapeutic amount is administered buccally. The related terms and phrases “administering” and “administration of,” when used in connection with a compound or tablet (and grammatical equivalents) refer both to direct administration, which may be administration to a patient by a medical professional or by self-administration by the patient, and/or to indirect administration, which may be the act of prescribing a drug. Administration entails delivery to the patient of the drug.

The term “dose” or “dosage” refers to the total amount of an active agent (e.g., osanetant or a pharmaceutically acceptable salt thereof) administered to a patient in a single day (24-hour period). The desired dose can be administered once daily. In some embodiments, the desired dose may be administered in one, two, three, four or more sub-doses at appropriate intervals throughout the day, where the cumulative amount of the sub-doses equals the amount of the desired dose administered in a single day. The terms “dose” and “dosage” are used interchangeably herein.

As used herein, “effective amount,” “therapeutically effective amount,” or “therapeutic amount” refers to an amount of a drug or an agent (e.g., osanetant or a pharmaceutically acceptable salt thereof) that when administered to a patient suffering from a condition, will have the intended therapeutic effect, e.g., alleviation, amelioration, palliation or elimination of one or more manifestations of the condition in the patient. The full therapeutic effect does not necessarily occur by administration of one dose, and can occur only after administration of a series of doses and can be administered in one dose form or multiples thereof. For example, 500 mg of the drug can be administered in a single 500 mg strength tablet or two 250 mg strength tablets. Thus, a therapeutically effective amount may be administered in one or more administrations.

As used herein, the term “patient” or “subject” refers to a mammal, such as a human, bovine, rat, mouse, dog, monkey, ape, goat, sheep, cow, or deer. A patient as described herein can be a human. The patient can be a male or a female.

As used herein, “treatment,” “treating,” and “treat” are defined as acting upon a disease, disorder, or condition with an agent to reduce or ameliorate the harmful or any other undesired effects of the disease, disorder, or condition and/or its symptoms. Treatment, as used herein, covers the treatment of a human patient, and includes: (a) reducing the risk of occurrence of the condition in a patient determined to be predisposed to the disease but not yet diagnosed as having the condition, (b) impeding the development of the condition, and/or (c) relieving the condition, i.e., causing regression of the condition and/or relieving one or more symptoms of the condition.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

### Methods

One of the most broadly experienced symptoms in women and men who have undergone hormone deprivation therapy, and/or medical or surgical interventions relating to cancer or prevention of cancer is vasomotor symptoms (VMS). In some instances, this is referred to as induced VMS or iVMS. VMS, such as hot flashes and night sweats, is characterized by a sudden feeling of warmth, usually localized in the face, neck, and chest, and is accompanied by sweating and flushing of the skin. The discomfort associated with these symptoms can negatively impact sufferers' ability to achieve restful sleep, and generally diminish quality of life.

Provided herein is a method of preventing, including blocking, attenuating, or limiting, the development of one or more vasomotor symptoms (VMS) in a patient, wherein the patient will be undergoing hormone deprivation therapy, a medical and/or surgical procedure that may cause VMS, comprising administering an effective amount of a neurokinin receptor antagonist, for a time period prior to, or concurrently with, the hormone deprivation therapy, a medical and/or surgical procedure. It is contemplated that, in certain embodiments, treating with NK3 antagonist prior to the surgery or hormone deprivation therapy will prevent the hypertrophy of the KNDy neurons, thereby leading to a lower effective dose of the NK3 compared to post-menopausal VMS. In some embodiments, blocking, attenuating, or limiting the development of one or more vasomotor symptoms comprises treating one or more of flushing of the skin, sweating, palpitations, racing heart rate, shivering, hot flashes, chills, irritability, anxiety, mood disorders, or depression. In some embodiments, the one or more vasomotor symptoms is hot flashes.

In certain embodiments, the patient has cancer, has had cancer, or has an increased risk for cancer. In some embodiments, the cancer is breast cancer, ovarian cancer, uterine cancer, testicular, or prostate cancer. In some embodiments, the cancer is breast cancer. In some embodiments, the cancer is metastatic breast cancer. In some embodiments, the cancer is ovarian cancer. In some embodiments, the cancer is uterine cancer. In some embodiments, the cancer is prostate cancer. In some embodiments, the cancer is hormone receptor-positive cancer, e.g., breast cancer or prostate cancer.

In some embodiments, the patient has suffered from cancer but is in remission. In some embodiments, the patient has an increased risk for cancer. In some embodiments, such a risk may be from a genetic mutation implicated in cancer. In some embodiments, the patient has tested positive for a BRCA1, BRCA2, or PALB2 mutation. In some embodiments, the patient has tested positive for a BRCA1 mutation. In some embodiments, the patient has tested positive for a BRCA2 mutation. In some embodiments, the patient has tested positive for a PALB2 mutation. In some embodiments, the patient is menopausal. In some embodiments, the patient is perimenopausal.

Inherited mutations to certain genes can lead to the development of specific types of cancers. BRCA1 and BRCA2 are genes that code for tumor suppressor proteins, which assist in repairing damage to DNA. When an individual has a mutated version of either gene, the individual will not produce a functional form of the related tumor suppressor protein, so DNA damage will accumulate, which can lead to the development of cancer. Mutations in BRCA1 and BRCA2 have been shown to increase the risk for the development of ovarian and fallopian tube cancer in women, prostate cancer in men, and, most notably, breast cancer in both women and men. In addition to BRCA1 and 2 mutations, patients having a mutation in PALB2 are also at risk for increase of cancer. In some embodiments provided herein, the genetic predisposition to developing certain mutation-linked or hormone-dependent cancers is having tested positive for a BRCA1 mutation. In some embodiments provided herein, the genetic predisposition to developing certain mutation-linked or hormone-dependent cancers is having tested positive for a BRCA2 mutation. In some embodiments provided herein, the genetic predisposition to developing certain mutation-linked or hormone-dependent cancers is having tested positive for a PALB2 mutation.

Given the significant increase in risk in developing these specific cancers associated with possessing a mutant BRCA1, BRCA2 or PALB2 gene, many women who are carriers opt for the prophylactic removal of breast tissue (mastectomy), fallopian tubes (salpingectomy), uterus (hysterectomy) and/or ovaries (oophorectomy), to stem the possibility of developing an associated cancer in these organs, as well as to limit the presence of the endogenous sex hormones that cause the proliferation of hormone-dependent cancers. In certain embodiments, the patient to be treated possess the BRCA1, BRCA2 or PALB2 gene and are having their breast tissue (mastectomy), fallopian tubes (salpingectomy), uterus (hysterectomy) and/or ovaries (oophorectomy) removed.

For men suffering from prostate cancer, surgical resection of the prostate (prostatectomy), seminal vesicles, and neighboring lymph nodes, as well as androgen suppression or deprivation therapy are common treatments for the disease. In situations of advanced prostate cancer, removal of the testes (orchiectomy), the primary source of endogenous testosterone in the male body, can be undertaken to further limit the growth and spread of the hormone-dependent prostate cancer.

For hormone-dependent cancers, such as breast, ovarian, uterine, prostate, and testicular cancers, the proliferation of cells is driven by hormone-receptor interactions on cell surfaces. In the presence of these sex hormones, namely estrogen, progesterone, and testosterone, the hormone-dependent cells replicate more frequently, increasing the opportunity for genetic errors to occur and accumulate, potentially leading to cancer. Pharmaceutical interventions, e.g. hormone deprivation therapy, for the treatment or prevention of hormone-dependent cancers include compounds that inhibit the synthesis of these sex hormones, such as gonadotropin-releasing hormone (GnRH) agonists and antagonists, compounds which block receptor sites on hormone-dependent cancer cell surfaces, such as selective estrogen-receptor modulators (SERMs) or nonsteroidal antiandrogens (NSAAs), and selective estrogen receptor degraders (SERDS). In certain embodiments, the patient will be undergoing hormone deprivation therapy. For instance, in the case of prostate cancer, the patient may be undergoing androgen deprivation therapy.

Many patients opt for medical or surgical interventions to remove the cancer and surrounding tissues. Many patients also elect for the removal of sex hormone producing organs, namely the ovaries or testes. In certain embodiments, the patient to be treated will be undergoing removal of ovaries or testes. In certain embodiments, the patient will already be receiving a GnRH agonist or antagonist, e.g. leuprolide. In certain embodiments, the patient will already be receiving a SERM, e.g. tamoxifen.

In some embodiments, the hormone deprivation therapy, and/or medical or surgical procedure that may cause VMS is the prophylactic removal of breast tissue (mastectomy). In some embodiments, the hormone deprivation therapy, and/or medical or surgical procedure that may cause VMS is the prophylactic removal of the fallopian tubes (salpingectomy). In some embodiments, the hormone deprivation therapy, and/or medical or surgical procedure that may cause VMS is the prophylactic removal of the ovaries (oophorectomy). In some embodiments, the hormone deprivation therapy, and/or medical or surgical procedure that may cause VMS is the prophylactic removal of one or more of breast tissue, fallopian tubes, and/or ovaries.

In some embodiments, the hormone deprivation therapy, and/or medical or surgical procedure that may cause VMS is the removal of the prostate (prostatectomy). In some embodiments, the hormone deprivation therapy, and/or medical or surgical procedure that may cause VMS is the removal of the seminal vesicles. In some embodiments, the hormone deprivation therapy, and/or medical or surgical procedure that may cause VMS is the removal of the testes (orchiectomy). In some embodiments, the hormone deprivation therapy, and/or medical or surgical procedure that may cause VMS is the administration of antiandrogen drugs. In some embodiments, the hormone deprivation therapy, and/or medical or surgical procedure that may cause VMS is the removal of the prostate, seminal vesicles, one or more testes, and/or the administration of antiandrogen drugs.

For decades, hormone replacement therapy (HRT), in which patients are given estrogen or an estrogen-progestin combination, has been prescribed to women to help ease their menopausal symptoms, including VMS. However, studies showing women who were treated with HRT for their menopausal symptoms, including VMS, had a higher incidence of certain hormone-dependent cancers, which has led medical professionals to reevaluate the risks associated with the practice. In patients who are already at an increased genetic risk for developing certain cancers, such as those with BRCA1 or BRCA2 mutations who may have undergone prophylactic surgeries to avoid the development of cancer in the previous mentioned organs, the risks associated with HRT treatment for VMS heavily outweigh the potential benefits. As such, in certain embodiments, HRT is contraindicated.

In some embodiments provided herein, the hormone therapy is estrogen therapy. In some embodiments provided herein, the hormone therapy is an estrogen and progestin combination therapy. In some embodiments provided herein, the hormone therapy is tibolone therapy.

In some embodiment the effective amount of osanetant, or a pharmaceutically acceptable salt thereof, is less than about 400 mg per day. In some embodiments, the effective amount of osanetant, or a pharmaceutically acceptable salt thereof, is from about 10 to about 350 mg per day. In some embodiments, the effective amount of osanetant, or a pharmaceutically acceptable salt thereof, is less than about 200 mg per day In some embodiments, the effective amount of osanetant, or a pharmaceutically acceptable salt thereof, is from about 10 to about 150 mg per day. In some embodiments, the effective amount of osanetant, or a pharmaceutically acceptable salt thereof, is about 300 mg per day. In some embodiments, the osanetant is administered once a day. In some embodiments, the osanetant, or a pharmaceutically acceptable salt thereof, is administered twice a day, each dose being about 150 mg.

In some embodiments, osanetant is dosed for a short period prior to surgery, or initiation of treatment with leuprolide or tamoxifen. In some embodiments, osanetant is administered for less than one week prior to surgery, or initiation of treatment with leuprolide or tamoxifen. In some embodiments, osanetant is administered for 1-3 days prior to surgery, or initiation of treatment with leuprolide or tamoxifen. In some embodiments, osanetant is administered for 1-2 days prior to surgery, or initiation of treatment with leuprolide or tamoxifen. In some embodiments, osanetant is administered on the day of, but prior to surgery, or initiation of treatment with leuprolide or tamoxifen. In some embodiments, osanetant is dosed for a short period prior to surgery, or initiation of treatment with leuprolide or tamoxifen and also concurrently with the surgery or treatment with leuprolide or tamoxifen. In some embodiments, osanetant is dosed for a short period prior to surgery, or initiation of treatment with leuprolide or tamoxifen, concurrently with the surgery or treatment with leuprolide or tamoxifen and continued after surgery or cessation of treatment with leuprolide or tamoxifen.

In some embodiments, for any method described herein, hormone therapy for the patient is contraindicated.

In some embodiments, the hormone therapy is estrogen therapy. In some embodiments, the hormone deprivation therapy is treatment with a selective estrogen receptor modulator (SERM). In some embodiments, the SERM is tamoxifen.

In some embodiments, the patient is a female patient. In some embodiments, the patient is a post-menopausal female patient.

In some embodiments, the hormone deprivation therapy is treatment with a gonadotropin-releasing hormone (GnRH) agonist or antagonist. In some embodiments, the patient is a male patient. In some embodiments, the GnRH agonist is leuprolide.

In some embodiments, the hormone deprivation therapy is treatment with a selective estrogen receptor degrader (SERD).

In some embodiments, the cancer is breast cancer, ovarian cancer, uterine cancer, or prostate cancer. In some embodiments, the cancer is hormone receptor-positive cancer. In some embodiments, the cancer is breast cancer. In some embodiments, the cancer is prostate cancer. In some embodiments, the patient has tested positive for a BRCA1, BRCA2, or PALB2 mutation.

In some embodiments, provided herein is a method for reducing the frequency and severity of hormone deprivation therapy-induced vasomotor symptoms or surgery-induced vasomotor symptoms in a cancer patient, the method comprising administering a combination of a hormone antagonist and an NK antagonist to the cancer patient in need thereof, wherein the NK antagonist is administered twice a day, each dose comprising from about 100 mg to about 200 mg of the NK antagonist. In some embodiments, the NK antagonist is a NK3 antagonist. In some embodiments, the NK3 antagonist is osanetant, or a pharmaceutically acceptable salt thereof. In some embodiments, the cancer patient is a BRCA1/2 positive breast cancer patient. In some embodiments, the NK antagonist is administered twice a day, each dose comprising about 150 mg of the NK antagonist.

In some embodiments, provided herein is a method for reducing the frequency and severity of tamoxifen-induced vasomotor symptoms or surgery-induced vasomotor symptoms in a cancer patient, the method comprising administering a combination of tamoxifen and an NK antagonist to the cancer patient in need thereof, wherein the NK antagonist is administered twice a day, each dose comprising from about 100 mg to about 200 mg of the NK antagonist. In some embodiments, the NK antagonist is a NK3 antagonist. In some embodiments, the NK3 antagonist is osanetant, or a pharmaceutically acceptable salt thereof. In some embodiments, the cancer patient is a BRCA1/2 positive breast cancer patient. In some embodiments, the NK antagonist is administered twice a day, each dose comprising about 150 mg of the NK antagonist.

Provided herein is a method for reducing leuprolide-induced vasomotor symptoms or surgery-induced vasomotor symptoms in a cancer patient, the method comprising administering a combination of leuprolide and an NK antagonist to the cancer patient in need thereof, wherein the NK antagonist is administered twice a day, each dose comprising from about 100 mg to about 200 mg of the NK antagonist. In some embodiments, the NK antagonist is a NK3 antagonist. In some embodiments, the NK3 antagonist is osanetant, or a pharmaceutically acceptable salt thereof. In some embodiments, the cancer patient is a prostate cancer patient. In some embodiments, the NK antagonist is administered twice a day, each dose comprising about 150 mg of the NK antagonist.

Provided herein is a method for reducing the frequency and severity of tamoxifen-induced vasomotor symptoms or surgery-induced vasomotor symptoms in a cancer patient, the method comprising administering a combination of tamoxifen and an NK antagonist to the cancer patient in need thereof, wherein the NK antagonist is administered twice a day, each dose comprising from about 25 mg to about 100 mg of the NK antagonist. In some embodiments, the NK antagonist is a NK3 antagonist. In some embodiments, the NK3 antagonist is osanetant. In some embodiments, the NK3 antagonist is osanetant, or a pharmaceutically acceptable salt thereof. In some embodiments, the cancer patient is a breast cancer patient. In some embodiments, the cancer patient is a BRCA1/2 positive or HR positive breast cancer patient. In some embodiments, the NK antagonist is administered twice a day, and the total daily dose of the NK antagonist ranges from about 50 mg per day to about 200 mg per day. In some embodiments, the dose is 25 mg administered twice a day. In some embodiments, the dose is 100 mg administered twice daily. In some embodiments, the NK antagonist is administered prior to initiation of tamoxifen treatment or prior to surgery for a period of less than one week (e.g., 1-6 days, 1-5 days, 1-4 days, 1-3 days, 1-2 days, or 1 day). In some embodiments, the NK antagonist is administered prior to initiation of tamoxifen treatment or prior to surgery for a period of one week. In some embodiments, the NK antagonist is administered prior to but on the day of initiation of tamoxifen treatment or surgery.

Provided herein is a method for reducing leuprolide-induced vasomotor symptoms or surgery-induced vasomotor symptoms in a cancer patient, the method comprising administering a combination of leuprolide and an NK antagonist to the cancer patient in need thereof, wherein the NK antagonist is administered twice a day, each dose comprising from about 25 mg to about 100 mg of the NK antagonist. In some embodiments, the dose is 25 mg administered twice a day. In some embodiments, the dose is 100 mg administered twice daily. In some embodiments, the NK antagonist is a NK3 antagonist. In some embodiments, the NK3 antagonist is osanetant. In some embodiments, the NK3 antagonist is osanetant, or a pharmaceutically acceptable salt thereof. In some embodiments, the cancer patient is a prostate cancer patient. In some embodiments, the NK antagonist is administered twice a day, and the total daily dose of the NK antagonist ranges from about 50 mg per day to about 200 mg per day. In some embodiments, the dose is 25 mg administered twice a day. In some embodiments, the dose is 100 mg administered twice daily. In some embodiments, the NK antagonist is administered prior to initiation of leuprolide treatment or prior to surgery for a period of less than one week (e.g., 1-6 days, 1-5 days, 1-4 days, 1-3 days, 1-2 days, or 1 day). In some embodiments, the NK antagonist is administered prior to initiation of leuprolide treatment or prior to surgery for a period of one week. In some embodiments, the NK antagonist is administered prior to but on the day of initiation of leuprolide treatment or surgery.

Provided herein is a method for reducing the frequency and severity of vasomotor symptoms in a patient undergoing bilateral salpingo-oophorectomy, the method comprising administering an NK antagonist to the patient in need thereof, wherein the NK antagonist is administered twice a day, each dose comprising from about 25 mg to about 100 mg of the NK antagonist. In some embodiments, the NK antagonist is a NK3 antagonist. In some embodiments, the NK3 antagonist is osanetant. In some embodiments, the NK3 antagonist is osanetant, or a pharmaceutically acceptable salt thereof. In some embodiments, the patient undergoing bilateral salpingo-oophorectomy is a breast cancer patient. In some embodiments, the NK antagonist is administered twice a day, and the total daily dose of the NK antagonist ranges from about 50 mg per day to about 200 mg per day. In some embodiments, the dose is 25 mg administered twice a day. In some embodiments, the dose is 100 mg administered twice daily. In some embodiments, the NK antagonist is administered prior to bilateral salpingo-oophorectomy for a period of less than one week (e.g., 1-6 days, 1-5 days, 1-4 days, 1-3 days, 1-2 days, or 1 day). In some embodiments, the NK antagonist is administered prior to bilateral salpingo-oophorectomy for a period of one week. In some embodiments, the NK antagonist is administered prior to but on the day of bilateral salpingo-oophorectomy.

In some embodiments, the neurokinin receptor antagonist is administered to a patient for a time period prior to the hormone deprivation therapy, and/or medical or surgical procedure as described herein. In some embodiments, the NK antagonist is administered concurrently with hormone deprivation therapy, a medical and/or surgical procedure. In some embodiments, the patient continues to receive a NK antagonist after the hormone deprivation therapy, a medical and/or surgical procedure. In some embodiments, the patient receives a NK antagonist after short-term (e.g., 1 to 6 months, 1 to 3 months) hormone deprivation therapy. Any combination of these therapeutic regimens is contemplated within the scope of embodiments presented herein.

In some embodiments, the administration of a neurokinin receptor antagonist confers an additional benefit and reduces or eliminates social isolation stress (SIS) in a cancer patient, thereby improving prognosis (e.g., lifespan, regression of the cancer, and/or quality of life) for cancer patients. Accordingly in any embodiment of any method described above and herein, the method further provides for alleviation of social isolation stress (SIS) in the cancer patient.

### NK Antagonists

As discussed above, NK antagonists are useful in the methods described herein. As used herein, “NK receptor,” “neurokinin receptor,” or “tachykinin receptor” is a transmembrane G-protein coupled receptor. The three known tachykinin receptors are NK1, NK2, and NK3. These receptors act on a variety of human functions, which regulate numerous biological systems, including the reproductive system.

As used herein, “NK receptor antagonists,” “neurokinin receptor antagonists,” or “tachykinin receptor antagonists” are a class of drugs which interact with tachykinin receptors NK1, NK2, and NK3, and dampen the normal agonist-mediated biological responses. The tachykinin receptors have been associated with the transmission of stress signals and pain, the contraction of smooth muscles, inflammation, and modulating the hypothalamus-pituitary-gonadal axis. NK receptor antagonists are indicated for the treatment of migraine, emesis, gastrointestinal disorders, disorders of the reproductive system, and psychiatric disorders, including anxiety, addiction, depression, and schizophrenia. In certain embodiments, the NK antagonist is a NK1, NK2, or NK3 antagonist or a combination thereof.

In certain embodiments, the NK antagonist is a NK1 receptor antagonists in selected from aprepitant, casopitant, ezlopitant, fosaprepitant, lanepitant, maropitant, rolapitant, vestipitant, L-733,060, L-741,671, L-742,694, RP-67580, RPR-100,893, CP-96345, CP-99994, GR-205,171, TAK-637, T-2328, and combinations thereof. In certain embodiments, the NK antagonist is a NK2 receptor antagonists selected from ibodutant, saredutant, GR-159,897, MEN-10376, and combinations thereof. In certain embodiments, the NK antagonist is a NK3 receptor antagonists selected from fezolinetant, osanetant, pavinetant, talnetant, (S)-3-methyl-2-phenyl-N-(1-phenylpropyl)-4-quinolinecarboxamide (SB-222,200, structure below), (−)-(R)—N-(α-methoxycarbonylbenzyl)-2-phenylquinoline-4-carboxamide (SB-218,795, structure below), and 2-[3,5-bis(trifluoromethyl)phenyl]-N-{4-(4-fluoro-2-methylphenyl)-6-[(7S,9aS)-7-(hydroxymethyl)hexahydropyrazino[2,1-c][1,4]oxazin-8(1H)-yl]pyridin-3-yl}-N,2-dimethylpropanamide (NT-814, structure below), and combinations thereof.

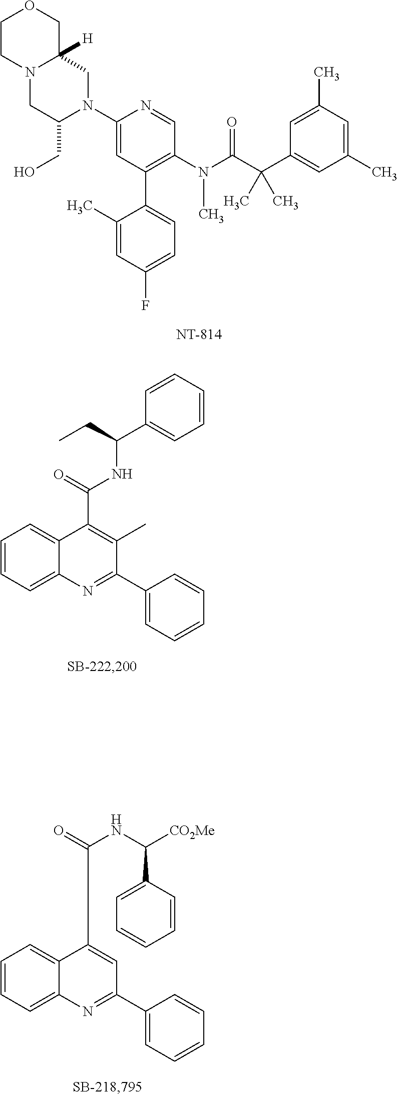

In certain embodiments, the NK antagonist is a NK3 antagonist. The NK3 receptor, and its associated tachykinin neuropeptide, neurokinin B, act on a variety of human functions, affecting the hypothalamus-pituitary-gonadal axis, which regulates numerous biological systems, including the reproductive system. In certain embodiments, the NK3 antagonist is osanetant.

In certain embodiments, the neurokinin-3 receptor antagonist is selected from osanetant, fezolinetant, pavinetant, talnetant, SB-222,200, SB-218,795, and NT-814. In certain embodiments, the neurokinin-3 receptor antagonist is osanetant. In certain embodiments, the neurokinin-3 receptor antagonist is fezolinetant. In certain embodiments, the neurokinin-3 receptor antagonist is pavinetant. In certain embodiments, the neurokinin-3 receptor antagonist is talnetant. In certain embodiments, the neurokinin-3 receptor antagonist is SB-222,200. In certain embodiments, the neurokinin-3 receptor antagonist is SB-218,795. In certain embodiments, the neurokinin-3 receptor antagonist is NT-814. In certain embodiments, the NK3 antagonist is osanetant.

Osanetant was originally developed for the treatment of schizophrenia and other central nervous system disorders. In certain embodiments the NK antagonist is osanetant or a stereoisomer, mixture of stereoisomers, prodrug, pharmaceutically acceptable salt, hydrate, solvate, acid salt hydrate, N-oxide or isomorphic crystalline form thereof. The chemical name of osanetant is (R)—N-(1-(3-(1-benzoyl-3-(3,4-dichlorophenyl)piperidin-3-yl)propyl)-4-phenylpiperidin-4-yl)-N-methylacetamide, and has the following structure:

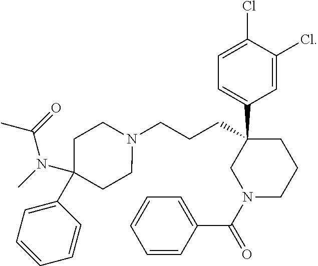 [SMILES: CC(=O)N(C)C1(c2ccccc2)CCN(CCC[C@]2(c3ccc(Cl)c(Cl)c3)CCCN(C(=O)c3ccccc3)C2)CC1]

Osanetant can also form pharmaceutically acceptable salts, such as osanetant hydrochloride, osanetant hydrobromide, osanetant sulfate, osanetant hydrogen sulfate, osanetant dihydrogen phosphate, osanetant methanesulfonate, osanetant methyl sulfate, osanetant maleate, osanetant fumarate, osanetant 2-naphthalenesulfonate, osanetant benzenesulfonate, osanetant glycolate, osanetant gluconate, and osanetant citrate, osanetant isethionate, osanetant p-toluenesulfonate, and the like. In some embodiments provided herein, osanetant is administered as a hydrochloride salt thereof.

Osanetant, as well as pharmaceutically acceptable salts thereof, can be purchased from commercial sources or can synthesized using published procedures.

### Hormone Deprivation Therapy

In some embodiments, the hormone deprivation therapy comprises treatment with a SERM as described throughout, e.g. tamoxifen. Women who have severe VMS are less likely to adhere to the tamoxifen treatment due to the hot flashes associated with the therapy. VMS typically decreases after cessation of tamoxifen treatment. Both hot flashes and sleep disturbances typically decrease after stopping tamoxifen. In one embodiment, the patient is female and optionally post-menopausal who is about to begin or is currently on a SERM therapy, e.g. tamoxifen.

In some embodiments, the hormone deprivation therapy comprises treatment with a gonadotropin-releasing hormone (GnRH) agonist or antagonist, e.g., leuprolide. In some embodiments, the patient is a male patient. As with tamoxifen, vasomotor symptoms are a common side effect in patients treated with leuprolide. Hot flashes occur in a majority of patients and can be severe. Hot flashes may reduce the quality of life in these patients and cause them to discontinue treatment.

In some embodiments, the hormone deprivation therapy is treatment with a selective estrogen receptor degrader (SERD). Examples of SERDs include and are not limited to

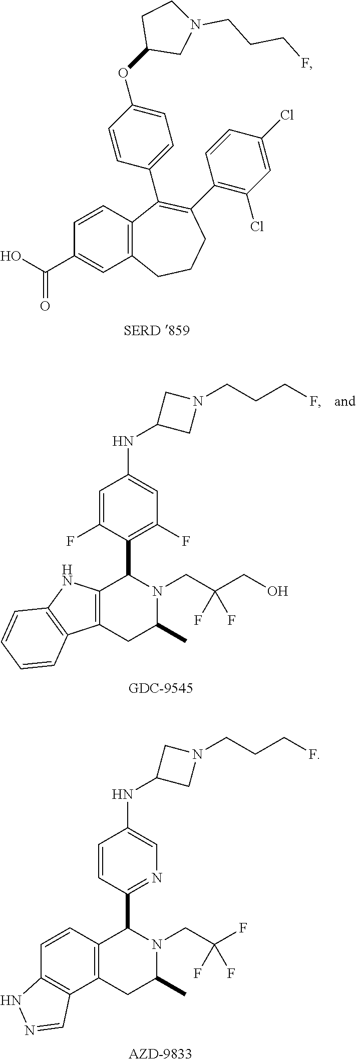

Vasomotor symptoms are a common side effect in patients treated with SERDs.

### Administration

In some embodiments, the time period over which the neurokinin receptor antagonist is administered to a patient prior to the hormone deprivation therapy, and/or medical or surgical procedure that may cause VMS is about 12 weeks or 8 weeks or 4 weeks to the day of the hormone deprivation therapy, and/or medical or surgical procedure. In some embodiments, the time period over which the neurokinin receptor antagonist is administered to a patient prior to the hormone deprivation therapy, and/or medical or surgical procedure is about 3 weeks to the day of the hormone deprivation therapy, and/or medical or surgical procedure. In some embodiments, the time period over which the neurokinin receptor antagonist is administered to a patient prior to the hormone deprivation therapy, and/or medical or surgical procedure that may cause VMS is about 2 weeks to the day of the hormone deprivation therapy, and/or medical or surgical procedure. In some embodiments, the time period over which the neurokinin receptor antagonist is administered prior to the hormone deprivation therapy, and/or medical or surgical procedure is about 2 weeks. In some embodiments, the time period over which the neurokinin receptor antagonist is administered prior to the hormone deprivation therapy, and/or hormone deprivation therapy, and/or medical or surgical procedure is about 1 week.

In some embodiments, the neurokinin receptor antagonist is administered to a patient concurrently with the hormone deprivation therapy, and/or medical or surgical procedure that may cause VMS.

In some embodiments, the time period over which the neurokinin receptor antagonist is administered to a patient after the hormone deprivation therapy, and/or medical or surgical procedure that may cause VMS is about 12 weeks. In some embodiments, the time period over which the neurokinin receptor antagonist is administered to a patient after the hormone deprivation therapy, and/or medical or surgical procedure that may cause VMS is about 8 weeks. In some embodiments, the time period over which the neurokinin receptor antagonist is administered to a patient after the hormone deprivation therapy, and/or medical or surgical procedure that may cause VMS is about 4 weeks.

In some embodiments, the patient is administered osanetant for about 1 week prior to, concurrently with, and for about 12 weeks after, a hormone deprivation therapy, and/or medical or surgical procedure. In some embodiments, the patient is administered osanetant for about 2 weeks prior to, concurrently with, and for about 12 weeks or more after, a hormone deprivation therapy, and/or medical or surgical procedure.

In some embodiments, the patient is administered osanetant or a stereoisomer, mixture of stereoisomers, prodrug, pharmaceutically acceptable salt, hydrate, solvate, acid salt hydrate, N-oxide or isomorphic crystalline form thereof, for about 1 week prior to, concurrently with, and for about 8 weeks after, a hormone deprivation therapy, and/or medical or surgical procedure. In some embodiments, the patient is administered osanetant for about 2 weeks prior to, concurrently with, and for about 8 weeks after, a hormone deprivation therapy, and/or medical or surgical procedure.

In some embodiments, the neurokinin receptor antagonist is orally administered.

In some embodiments, the neurokinin receptor antagonist is intranasally administered.

In some embodiments, the neurokinin receptor antagonist is subcutaneously administered.

In some embodiments, the neurokinin receptor antagonist is transdermally administered.

In some embodiments, the neurokinin receptor antagonist is intravenously administered.

In some embodiments, the neurokinin receptor antagonist is buccally administered.

In some embodiments, the neurokinin receptor antagonist is administered once daily. In some embodiments, the neurokinin receptor antagonist is administered as two, three, four or more sub-doses at appropriate intervals throughout the day, where the cumulative amount of the sub-doses equals the amount of the desired dose administered in a single day.

In some embodiments, when the NK antagonist is osanetant or a stereoisomer, mixture of stereoisomers, prodrug, pharmaceutically acceptable salt, hydrate, solvate, acid salt hydrate, N-oxide or isomorphic crystalline form thereof, the therapeutically effective amount of the osanetant, or a stereoisomer, mixture of stereoisomers, prodrug, pharmaceutically acceptable salt, hydrate, solvate, acid salt hydrate, N-oxide or isomorphic crystalline form thereof, is about 0.25 mg/day to about 1000 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, osanetant is about 0.5 mg/day to about 500 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 0.75 mg/day to about 450 mg/day, is about 1 mg/day to about 400 mg/day or 10 mg/day to about 350 mg/day. In some embodiments, the osanetant is administered is less than about 400 mg/day. In some embodiments, the osanetant is administered is less than about 200 mg/day or about 10 mg/day to about 150 mg/day.

In some embodiments, the therapeutically effective amount of the osanetant, or a stereoisomer, mixture of stereoisomers, prodrug, pharmaceutically acceptable salt, hydrate, solvate, acid salt hydrate, N-oxide or isomorphic crystalline form thereof, is about 1 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 50 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 100 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 200 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 300 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 400 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 500 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 600 mg/day. In some embodiments, the therapeutically effective amount of osanetant, or a stereoisomer, mixture of stereoisomers, prodrug, pharmaceutically acceptable salt, hydrate, solvate, acid salt hydrate, N-oxide or isomorphic crystalline form thereof, is about 800 mg/day. In some embodiments, the therapeutically effective amount of osanetant is about 1000 mg/day.

In certain embodiments, osanetant, or a stereoisomer, mixture of stereoisomers, prodrug, pharmaceutically acceptable salt, hydrate, solvate, acid salt hydrate, N-oxide or isomorphic crystalline form thereof, is dosed at about 0.25 mg/day to about 1 mg/day, in order to treat VMS symptoms while avoiding transient increases in liver transaminase (alanine aminotransferase) concentrations, and maintaining healthy liver function.

In some embodiments, osanetant is administered prior to, and/or concurrently with, and/or after administration of tamoxifen in a daily dose ranging from about 5 mg per day to about 600 mg per day. In some embodiments, osanetant is administered prior to, and/or concurrently with, and/or after administration of tamoxifen in a daily dose ranging from about 25 mg per day to about 500 mg per day. In some embodiments, osanetant is administered prior to, and/or concurrently with, and/or after administration of tamoxifen in a daily dose ranging from about 50 mg per day to about 500 mg per day. In some embodiments, osanetant is administered prior to, and/or concurrently with, and/or after administration of tamoxifen in a daily dose ranging from about 100 mg per day to about 400 mg per day. In some embodiments, osanetant is administered prior to, and/or concurrently with, and/or after administration of tamoxifen in a daily dose ranging from about 150 mg per day to about 300 mg per day. In any of these embodiments, the total daily dose may be administered as a single dose, or split between multiple doses (e.g., daily dose of 300 mg QD or daily dose of 300 mg split into 150 mg BID, or daily dose of 300 mg split into 100 mg TID). In some embodiments, osanetant is administered prior to, and/or concurrently with, and/or after administration of tamoxifen in a daily dose of 300 mg, split into two doses of 150 mg each, i.e., 150 mg BID. In some embodiments, osanetant is administered prior to, and/or concurrently with, and/or after administration of tamoxifen in a daily dose of 200 mg, split into two doses of 100 mg each, i.e., 100 mg BID. In some embodiments, osanetant is administered prior to, and/or concurrently with, and/or after administration of tamoxifen in a daily dose of 100 mg, split into two doses of 50 mg each, i.e., 50 mg BID. In some embodiments, osanetant is administered prior to, and/or concurrently with, and/or after administration of tamoxifen in a daily dose of 50 mg, split into two doses of 25 mg each, i.e., 25 mg BID. In some embodiments, osanetant is administered prior to, and/or concurrently with, and/or after administration of tamoxifen in a daily dose of 25 mg, split into two doses of 12.5 mg each, i.e., 12.5 mg BID.

In some embodiments, osanetant is administered prior to, and/or concurrently with, and/or after administration of leuprolide in a daily dose ranging from about 5 mg per day to about 600 mg per day. In some embodiments, osanetant is administered prior to, and/or concurrently with, and/or after administration of leuprolide in a daily dose ranging from about 25 mg per day to about 500 mg per day. In some embodiments, osanetant is administered prior to, and/or concurrently with, and/or after administration of leuprolide in a daily dose ranging from about 50 mg per day to about 500 mg per day. In some embodiments, osanetant is administered prior to, and/or concurrently with, and/or after administration of leuprolide in a daily dose ranging from about 100 mg per day to about 400 mg per day. In some embodiments, osanetant is administered prior to, and/or concurrently with, and/or after administration of leuprolide in a daily dose ranging from about 150 mg per day to about 300 mg per day. In any of these embodiments, the total daily dose may be administered as a single dose, or split between multiple doses (e.g., daily dose of 300 mg QD or daily dose of 300 mg split into 150 mg BID, or daily dose of 300 mg split into 100 mg TID). In some embodiments, osanetant is administered prior to, and/or concurrently with, and/or after administration of leuprolide in a daily dose of 300 mg, split into two doses of 150 mg each, i.e., 150 mg BID. In some embodiments, osanetant is administered prior to, and/or concurrently with, and/or after administration of leuprolide in a daily dose of 200 mg, split into two doses of 100 mg each, i.e., 100 mg BID. In some embodiments, osanetant is administered prior to, and/or concurrently with, and/or after administration of leuprolide in a daily dose of 100 mg, split into two doses of 50 mg each, i.e., 50 mg BID. In some embodiments, osanetant is administered prior to, and/or concurrently with, and/or after administration of leuprolide in a daily dose of 50 mg, split into two doses of 25 mg each, i.e., 25 mg BID. In some embodiments, osanetant is administered prior to, and/or concurrently with, and/or after administration of leuprolide in a daily dose of 25 mg, split into two doses of 12.5 mg each, i.e., 12.5 mg BID.

### Pharmaceutical Compositions

Also provided herein, in some embodiments, are pharmaceutical compositions that comprise osanetant, or a stereoisomer, mixture of stereoisomers, prodrug, pharmaceutically acceptable salt, hydrate, solvate, acid salt hydrate, N-oxide or isomorphic crystalline form thereof, and one or more pharmaceutically acceptable vehicles selected from carrier, adjuvants, and excipients.

Suitable pharmaceutically acceptable vehicles may include, for example, inert solid diluents and fillers, diluents, including sterile aqueous solutions and various organic solvents, permeation enhancers, solubilizers, and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal, intravenous, subcutaneous, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

The pharmaceutical composition may be in the form of a sterile injectable aqueous or oleaginous suspension. This suspension may be formulated according to the known art using suitable dispersing or wetting agents and suspending agents. The sterile injectable preparation may also be sterile injectable solution or suspension in a non-toxic parentally acceptable vehicle, for example as a solution in 1,3-butanediol. Among the acceptable vehicles that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid can be useful in the preparation of injectables. Such solutions may be formulated as 0.01%-10% isotonic solutions, pH 5-7, with appropriate salts.

The compound described herein may be administered parenterally in a sterile medium. Parenteral administration includes subcutaneous injections, intravenous, intramuscular, intrathecal injection or infusion techniques. The compound described herein, depending on the vehicle and concentration used, can either be suspended or dissolved in the vehicle. Advantageously, adjuvants such as local anesthetics, preservatives and buffering agents can be dissolved in the vehicle. In many pharmaceutical compositions for parenteral administration the carrier comprises at least 90% by weight of the total composition. In some embodiments, the carrier for parenteral administration is chosen from propylene glycol, ethyl oleate, pyrrolidone, ethanol, and sesame oil.

A pharmaceutical composition, for example, for injection, may comprise a cyclodextrin. The cyclodextrin may be, for example, a hydroxypropyl cyclodextrin or a sulfobutylether cyclodextrin. The cyclodextrin may be, for example, an α-cyclodextrin, a β-cyclodextrin, or a γ-cyclodextrin.

A compound described herein may also be administered via microspheres, liposomes, other microparticulate delivery systems or sustained release formulations placed in certain tissues including blood. Suitable examples of sustained release carriers include semi-permeable polymer matrices in the form of shared articles, e.g., suppositories or microcapsules. Examples can be found, e.g., in Remington's Pharmaceutical Sciences, 18th edition, Gennaro, A. R., Lippincott Williams & Wilkins; 20th edition (Dec. 15, 2000) ISBN 0-912734-04-3 and Pharmaceutical Dosage Forms and Drug Delivery Systems; Ansel, N. C. et al. 7th Edition ISBN 0-683305-72-7, the entire disclosures of which are herein incorporated by reference.

Oral administration may be another route for administration of the compounds described herein. Administration may be via, for example, capsule or enteric coated tablets. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The compound described herein can be incorporated into oral liquid preparations such as aqueous or oily suspensions, solutions, emulsions, syrups, or elixirs, for example. Furthermore, pharmaceutical compositions containing the compound described herein can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monooleate, or acacia), non-aqueous vehicles, which can include edible oils (e.g., almond oil, fractionated coconut oil, silyl esters, propylene glycol and ethyl alcohol), and preservatives (e.g., methyl or propyl p-hydroxybenzoate and sorbic acid).

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, orally or nasally, from devices that deliver the formulation in an appropriate manner.

Buccal administration, where the pharmaceutical composition is placed between the gum and cheek and diffuses through the oral mucosa, may be another route for administration of the compounds described herein. The forms in which the pharmaceutical compositions described herein may be incorporated for buccal administration include, for example, quick-dissolving tablets, buccal mucoadhesive tablets, lozenges, powders, sprays, mucoadhesive buccal patches and films, ointments, gels, or liquid suspensions. Formulations for the pharmaceutical compositions for buccal administration that include at least one compound described herein may also include mucoadhesive agents, to maintain prolonged contact of the formulation with the oral mucus membrane, penetration enhancers, to improve drug permeation across the oral mucus membrane, enzyme inhibitors, to protect the active ingredient from enzymatic degradation, and solubility modifiers. The active ingredient is also usually diluted by an excipient.

Some examples of suitable mucoadhesive agents include agarose, chitosan, trimethylated chitosan, chitosan-EDTA, gelatin, hyaluronic acid, guar gum, hakea gum, xanthan gum, gellan gum, carrageenan, pectin, sodium alginate, cellulose derivatives, CMC, thiolated CMC, sodium CMC, HEC, HPC, HPMC, MC, poly(acrylic acid)-based polymers, CP, PC, PAA, copolymers of acrylic acid and PEG, PVA, PVP, CP, aminodextran, dimethylaminoethyl-dextran, hydroxyethyl starch, poly(ethylene oxide), scleroglucan, cyanoacrylate, hydroxylated methacrylate, and poly(methacrylic acid). Some examples of suitable penetration enhancers include sodium lauryl sulfate, cetyl pyridinium chloride, Poloxamer, Brij, Span, Myrj, Tween, sodium glycocholate, sodium tauro deoxycholate, sodium tauro cholate, oleic acid, caprylic acid, lauric acid, lyso phosphatidyl choline, phosphatidyl choline, α-, β-, and γ-cyclodextrin, methylated β-cyclodextrin, EDTA, citric acid, sodium salicylate, methoxy salicylate, chitosan, trimethyl chitosan, poly-L-arginine, and L-lysine. Some examples of suitable enzyme inhibitors include aprotinin, bestatin, and puromycin.

### Combination Therapy

The neurokinin receptor antagonist may be administered in combination with one or more additional active agents. Thus, the methods described herein include methods for preventing, including blocking, attenuating, or limiting, the development of one or more vasomotor symptoms in a patient for which hormone therapy is contraindicated, wherein the patient will be undergoing a hormone deprivation therapy, and/or medical or surgical procedure that may cause VMS, comprising administering, simultaneously or sequentially, an effective amount of the neurokinin receptor antagonist and one or more additional active agent(s), for a time period prior to, or concurrently with, the hormone deprivation therapy, and/or medical or surgical procedure. In certain embodiments of the administration of a combination therapy including the neurokinin receptor antagonist, the patient has cancer, has had cancer, or is at an increased risk for cancer. In methods using simultaneous administration, the neurokinin receptor antagonist and the additional active agent(s) can be present in a combined composition or can be administered separately. When used in combination with one or more additional active agents, the neurokinin receptor antagonist may be administered prior to, concurrently with, or following administration of the additional active agents. The administration can be by the same route or by different routes. In some embodiments, the neurokinin receptor antagonist may be administered in combination with a second active agent. In some embodiments, the second active agent may be a SERM, SERD, GnRH or NSAA.

As used herein, “selective estrogen-receptor modulators” or “SERMs” are a class of drug which have varied estrogenic and antiestrogenic effects on estrogen receptors, depending on the tissue in which the receptors are located. This allows for selective estrogen receptor modulation in certain tissue types by choosing the appropriate SERM.

As used herein, “gonadotropin-releasing hormone agonists and antagonists” or “GnRH agonists and antagonists” are classes of drugs which prevent the GnRH-mediated release of sex hormones. GnRH is a peptide hormone produced by GnRH neurons in the hypothalamus, responsible for the release of follicle-stimulating hormone and luteinizing hormone from the pituitary gland, beginning the hypothalamic-pituitary-gonadal axis synthesis and release of sex hormones.

As used herein, “nonsteroidal antiandrogens” or “NSAAs” are a class of drug that are antagonists of androgen receptors, blocking the action of testosterone and dihydrotestosterone in tissue.

As used herein, “selective estrogen receptor degraders” or “SERDs” are a class of drug that binds to the estrogen receptor (ER) and, in the process of doing so, causes the ER to be degraded and thus downregulated.

In some embodiments, the second active agent may be a selective estrogen receptor modulator (SERM), including, but not limited to, anordrin (+mifepristone (Zi Yun)), bazedoxifene (+conjugated estrogens (Duavee)), broparestrol (Acnestrol), clomifene (Clomid), cyclofenil (Sexovid), lasofoxifene (Fablyn), ormeloxifene (Centron, Novex, Novex-DS, Sevista), ospemifene (Osphena; deaminohydroxytoremifene), raloxifene (Evista), tamoxifen (Nolvadex), toremifene (Fareston; 4-chlorotamoxifen), acolbifene, afimoxifene (4-hydroxytamoxifen; metabolite of tamoxifen), elacestrant, enclomifene ((E)-clomifene), endoxifen (4-hydroxy-N-desmethyltamoxifen; metabolite of tamoxifen), zuclomifene ((Z)-clomifene), arzoxifene, brilanestrant, clomifenoxide (clomiphene N-oxide; metabolite of clomifene), droloxifene (3-hydroxytamoxifen), etacstil, fispemifene, (E)-3-[4-[(E)-1-(4-hydroxyphenyl)-2-phenylbut-1-enyl]phenyl]prop-2-enoic acid (GW-7604) (4-hydroxyetacstil; metabolite of etacstil), idoxifene (pyrrolidino-4-iodotamoxifen), levormeloxifene ((L)-ormeloxifene), miproxifene, nafoxidine, nitromifene (CI-628), 4-(2-ethyl-11-azatricyclo[5.3.1.04,11]undeca-1(10),2,4,6,8-pentaen-3-yl)phenol (NNC 45-0095), panomifene, pipendoxifene (ERA-923), trioxifene, or zindoxifene (D-16726). In some embodiments, the second active agent may be a gonadotropin-releasing hormone agonist, including, but not limited to, buserelin, deslorelin, fertirelin, gonadorelin, goserelin, histrelin, lecirelin, leuprorelin, nafarelin, peforelin, triptorelin, abarelix, cetrorelix, degarelix, ganirelix, elagolix, or relugolix. In some embodiments, the second active agent may be a nonsteroidal antiandrogen (NSAA), including, but not limited to, flutamide, nilutamide, bicalutamide, topilutamide, apalutamide, enzalutamide, darolutamide, cimetidine, proxalutamide, seviteronel, cioteronel, inocoterone acetate, or 4-(3-(4-Hydroxybutyl)-4,4-dimethyl-2,5-dioxoimidazolidin-1-yl)-2-(trifluoromethyl)benzonitrile (RU-58841).

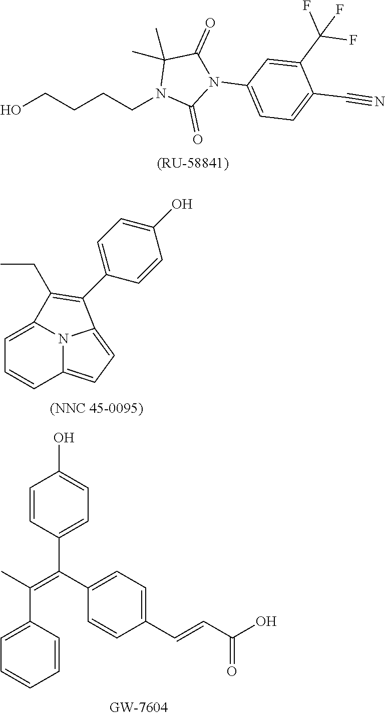 [SMILES: CC1(C)C(=O)N(c2ccc(C#N)c(C(F)(F)F)c2)C(=O)N1CCCCO ; CCc1c(-c2ccc(O)cc2)c2ccc3cccc1n32 ; C/C(=C(\c1ccc(O)cc1)c1ccc(/C=C/C(=O)O)cc1)c1ccccc1]

In some embodiments, the second active agent may be a selective estrogen receptor degrader (SERD), including, but not limited to, fulvestrant, brilanestrant, elacestrant, SERD '859, GDC-9545, and AZD-9833.

In some embodiments, the second active agent may be a selective androgen receptor degrader (SARD), including, but not limited to, dimethylcurcumin.

In one embodiment, the NK antagonist is administered with a kappa opioid agonist. In certain embodiments the kappa opioid receptor agonist is selected from alazocine, bremazocine, 8-carboxamidocyclazocine, cyclazocine, ketazocine, metazocine, pentazocine, phenazocine, 6′-guanidinonaltrindole (6′-GNTI), butorphan, butorphanol, cyclorphan, diprenorphine, etorphine, levallorphan, levomethorphan, levorphanol, morphine, nalbuphine, nalfurafine, nalmefene, nalodeine, nalorphine, norbuprenorphine, norbuprenorphine-3-glucuronide, oxilorphan, oxycodone, proxorphan, samidorphan, xorphanol, asimadoline, BRL-52537, eluxadoline, enadoline, GR-89696, ICI-204,448, ICI-199,441, LPK-26, MB-IC-OH, niravoline, N-MPPP, spiradoline, U-50,488, U-54,494A, U-69,593, CR665, difelikefalin (CR845), dynorphins (dynorphin A, dynorphin B, big dynorphin), collybolide, erinacine E, menthol, RB-64, salvinorin A, 2-methoxymethyl salvinorin B (and its ethoxymethyl and fluoroethoxymethyl homologues), apadoline, HS665, HZ-2, ibogaine, ketamine, noribogaine, tifluadom, and combinations thereof.

Also provided is a pharmaceutical composition comprising osanetant and a second active agent.

### EXAMPLES

It is understood that modifications which do not substantially affect the activity of the various embodiments of this disclosure are also included within the definition of the disclosure provided herein. Accordingly, the following examples are intended to illustrate but not limit the present disclosure.

### Example 1—Treatment with Osanetant

Women between the ages of 40 to 65 years old are confirmed as menopausal per one of the following criteria at the outset of the study: spontaneous amenorrhea for at least 12 consecutive months; spontaneous amenorrhea for at least 6 months with biochemical criteria of menopause (follicle-stimulating hormone [FSH]>40 IU/L); or having had bilateral oophorectomy at least 6 weeks prior to outset of study. Subjects must have a minimum average of 50 to 60 moderate to severe hot flashes per week. Subjects are administered osanetant, or a pharmaceutically acceptable salt thereof, orally and twice daily, or are administered a placebo control. Osanetant is administered to the treated group in a total daily dosage of between 100 and 400 mg. Vasomotor symptom (e.g. hot flash) frequency and severity are self-reported on a daily basis throughout a 12-week treatment period and a 2-week recovery period.

Additional subjects are studied, including women with HR positive breast cancer who are receiving tamoxifen, men with HR positive prostate cancer receiving leuprolide and women who are BRCA positive and have had bilateral oophorectomy.

### Example 2—Animal Model

Young adult, female Sprague-Dawley rats (approximately 12 wk old, 200-250 g; Harlan Laboratories) are individually housed in a quiet, temperature and humidity-controlled room (ambient temperature of 21.1-22.5° C., humidity set at 50%) with a 12:12 h light:dark cycle (lights on at 0700 h). Rats are given ad libitum access to water and a low-phytoestrogen diet. A Unified Information Device (PhysioTel transmitter TA10-F40; Data Sciences International, DSI) is inserted into the peritoneal cavity by IP injection for measurements of T_{CORE} and activity. Basal core and tail temperatures are recorded for 7 days (AM and PM).

Rats (n=24) are ovariectomised (OVX) under general anesthesia using a mixture (1.0 mL/kg i.m.) containing ketamine (33.3 mg/mL), xylazine (10.7 mg/mL), and acepromazine (1.3 mg/mL). Basal core and tail temperatures are recorded for 7 days (AM and PM) after surgery.

Rats are dosed orally twice each day with a suspension of osanetant in 0.6% methylcellulose or another NK3 antagonist. Basal core and tail temperatures are recorded at 10 minute intervals for 60 min following each dosing. Doses of 12 mg/kg/day, 61.5 mg/kg/day, 123 mg/kg/day, 184.5 mg/kg/day, and 250 mg/kg/day will be tested. Each total daily dose will be split into two equal doses. For instance, 12 mg/kg/day dose will be administered as two doses of 6 mg/kg/day.

Control: Twenty to 23 d after the initial surgery, under isoflurane anesthesia, rats are implanted with two s.c. capsules (each 20 mm effective length, 1.57 mm inner diameter, 3.18 mm outer diameter; Dow Corning) containing 360 μg/mL 17β-estradiol dissolved in sesame oil. Radioimmunoassay (RIA) is used to determine whether this regimen produces physiological levels of serum E₂ that are similar in control animals and NK3-treated animals.

Temperature Recordings. T_{CORE} and gross motor activity are measured via telemetry using the implanted DSI transmitter. Individual cages are placed on an RPC-1 Physiotel receiver that is connected by a Data Exchange Matrix (DSI) to a computer equipped with Dataquest A.R.T. software (DSI). TSKIN is recorded with a SubCue Mini datalogger (SubCue Dataloggers). The dataloggers are housed in a protective nylon casing taped to the lateral surface of the tail (4.0 cm from the base) under brief (<5 min) isoflurane anesthesia. T_{AMBIENT} is recorded with an IT-18 thermocouple (Physitemp) inserted into a QuadTemp datalogger (Madgetech). Temperature measurement devices are calibrated according to manufacturer specifications and validated against a National Institute of Standards and Technology certified TC4000 thermocouple datalogger (Madgetech).

Experiment 1: Effects of NK3 inhibition on Circadian Rhythms of T_{CORE}, T_{SKIN}, and Activity in OVX and OVX+17β-estradiol (E₂) Rats. Twelve to 15 d after administration of the NK3 inhibitor, T_{CORE}, T_{SKIN}, activity, and T_{AMBIENT} are recorded every 10 min for five consecutive 24-h light/dark cycles. One day after E₂ capsule implantation, these recordings are repeated for another five 24-h cycles. For these 5-d recording sessions, the rats are housed in their home cages in a dedicated room in the animal facility that is relatively isolated from noise. Cage cleaning is conducted before and after the 5-d recordings. The rats are placed in transparent, plastic shoebox-style cages containing wood-shaving bedding and the cages are placed on individual telemetry receivers. Although individual cages are needed for telemetry, the rats maintain visual, auditory, and olfactory contact with other animals.

Experiment 2: Effects of NK3 inhibition on the E₂ Modulation of T_{CORE}, T_{SKIN}, and HLI in Rats Exposed to T_{AMBIENT} of 26° C., 11° C., or 33° C. To determine if NK3 inhibition altered the ability of the rats to defend T_{CORE} in response to environmental temperature challenges, rats are exposed to temperatures that were within the thermoneutral zone (26° C.), subneutral (11° C.), or supraneutral (33° C.) T_{AMBIENT}. The thermoneutral zone is defined as the range of T_{AMBIENT} in which thermoregulation is achieved only by sensible (dry) heat loss, without regulatory changes in metabolic heat production or evaporative cooling. Within the thermoneutral zone, T_{CORE} is regulated primarily by skin vasomotion.

After the completion of the circadian rhythm recordings, animals are brought to the laboratory for three consecutive mornings and exposed to one of three T_{AMBIENT} in an environmental chamber. The experiments are conducted in the morning to avoid the confounding influence of E₂ positive feedback. Animals are habituated to the experimental procedure on three occasions within the first 14 d after surgery. The environmental chamber (Forma model 3940; Thermo Scientific) is equilibrated to the required temperature with humidity set at 50%. Each rat is placed in a 6 inch×6 inch×4 inch plastic grid cage, which allows free movement and ad libitum access to food and water. The cages are placed on telemetry receivers in the environmental chamber and T_{CORE}, T_{SKIN}, and T_{AMBIENT} are recorded every 10 min for 3 h. This procedure is repeated after the OVX rats are implanted with E₂ and a second set of circadian rhythm recordings is obtained.

For sacrifice, animals are injected with a lethal dose of sodium pentobarbital (100 mg/kg i.p.) and perfused through the ascending aorta with 200 mL of 0.1 M phosphate buffered heparinized saline followed by 400 mL of 4% (wt/vol) paraformaldehyde in 0.1 M PBS, pH 7.4. Optionally, brains are extracted and immunohistochemical methods are used to characterize the effects of NK3 inhibitor administration.

Data Analysis. A temperature probe on the tail skin surface will reflect not only active changes in vasomotor tone, but also passive changes in T_{AMBIENT} and T_{CORE}. To provide a more accurate assessment of tail skin vasomotion, the heat loss index [HLI=(T_{SKIN}−T_{AMBIENT})/(T_{CORE}−T_{AMBIENT})] is calculated, which removes the passive influences of ambient and core temperatures and can be correlated with blood flow. The theoretical range of the HLI is between 0 (corresponding to maximal skin vasoconstriction) and 1 (maximal skin vasodilatation).

Circadian rhythms are evaluated over the 5-d period using circadian physiology software. Averages of T_{SKIN}, T_{CORE}, HLI, and activity are calculated for each animal during light (inactive) or dark (active) phases. Six hour time blocks are used from the middle of each phase (1000 h to 1600 h and 2200 h to 0400 h for light and dark, respectively) to avoid the lights on/off transition. Because E₂ effects on T_{SKIN} during the dark phase are not expected to be significant until 3 d after capsule implantation, days 3-5 of the circadian recordings will be used for statistical analysis. Group averages of T_{SKIN}, T_{CORE}, activity (counts/6 h), and HLI will be generated from the means of individual rats. Group means will be compared using a two-way ANOVA with Tukey's post hoc analysis (α=0.05).

For the temperature challenges, the mean T_{CORE} and T_{SKIN} for each animal will be calculated from the second and third hour of recording in the environmental chamber. The averages from each animal will be used to calculate group averages.

Data will be analyzed using a two-way ANOVA and Tukey's post hoc tests (α=0.05). Normothermia (37.2±0.3° C.) is considered to be the average T_{CORE} (±1 SD) during the light phase of all rats exposed to a T_{AMBIENT} of 26° C., which is within the thermoneutral zone of both OVX and OVX+E₂ rats.

A similar experiment will be conducted for young adult, male Sprague-Dawley rats (approximately 12 wk old, 200-250 g).

### Example 3—Virtual Study

A virtual platform was used to study dosing methodologies and outcomes. In the platform, a quantitative systems pharmacology (QSP) model of the hypothalamus-pituitary-gonadal (HPG) axis was used to evaluate combinations with osanetant for treatment of induced VMS (HPG QSP model). The platform includes three specific biomarkers for estimating vasomotor symptoms: Neurokinin B, GnRH, and Vasomotor symptoms (VMS). The goal of the study was to optimize dosing strategies for combining osanetant with tamoxifen and leuprolide to reduce induced vasomotor symptoms without impacting anti-cancer efficacy. A secondary goal was to assess the potential of drug-drug interaction between osanetant and tamoxifen and leuprolide at the liver metabolism and drug transporter level.

Two Virtual Patients (VP) were developed for testing and research in the Platform: a Male VP and Postmenopausal Female VP based on certain Multidimensional Quality Metrics (MQM) criteria. The Male VP was used to research the effects of osanetant on leuprolide-induced VMS. The Postmenopausal VP was used to research the effects of osanetant on postmenopausal VMS and tamoxifen-induced VMS.

In the platform, VMS are estimated into two separate quantities: severity and frequency. These are based on measures from known clinical trials. Hot flashes are typically measured in a hot flash diary for frequency and severity (mild, moderate, severe) (Fisher, W. I., and R. C. Thurston. 2016. Menopause 23 (11):1222-1227).

In the platform, VMS severity represents the relative intensity of the VMS ranging from 0 to 10, where 10 is the most severe and 0 is no VMS. It is calculated as a linear correlation of bound NK3 receptor. It is calibrated for the baseline severity, response to cancer therapy, and response to osanetant. The differing baseline severity between an average postmenopausal female and male was accounted for by using different correlations for the patient types. The expected NKB levels and NK3 binding following therapies constrained the expected change in severity.

VMS frequency represents the expected number of hot flashes in a day ranging from 0 to 12 hot flashes per day. The occurrence of hot flashes from hour-to-hour is not represented. It is calculated as a linear correlation of bound NK3 receptor. It is calibrated for the baseline frequency, response to cancer therapy, and response to osanetant. The differing baseline frequency between an average postmenopausal female and male was accounted for by using different correlations for the patient types. The expected NKB levels and NK3 binding following therapies constrained the expected change in frequency. A placebo effect is included for VMS frequency. This was done because all clinical trials that provide VMS frequency data show a similar strong placebo effect. The placebo effect is dependent on the drug dose timing, as seen in the clinical data. In this modeling study, a placebo dose is administered concomitantly with osanetant to recreate the expected placebo effect in the first five weeks for both frequency and severity. The placebo affects the NK3 binding as a competitive antagonist, which is identical to osanetant.

The equations for these measures are

VMS_Severity=max(0,NK3R_VMS_Sev_k*(NK3R_Bound−NK3R_Bound_Initial))

VMS Frequency=max(0,NK3R_VMS_Freq_k*(NK3R_Bound−NK3R_Bound_Initial)−Placebo_Effect)

Placebo is a dose. Amount=0.07, Calibrated for −30% drop in frequency. Twice per day, 12 hours apart.

The model includes detailed NKB, dynorphin and estradiol effects on KNDy neurons, neuroendocrine feedback, and downstream effects on the HPG axis and sex hormones. Model development software: MATLAB® SimBiology®. Schmidt H, Jirstrand M. (2006) Bioinformatics 22, 514-5

### Tamoxifen

In the platform, tamoxifen is dosed at 40 mg daily. Tamoxifen pharmacokinetics include complex metabolism and multiple tissue compartments. In the platform, tamoxifen PK is implemented using data from multiple papers and based on multiple PK models. Data used to develop the tamoxifen pharmacokinetics section include: Population PK: (Dickschen K. S. et al. 2012, Front Pharmacol 3:92; ter Heine R. L. et al. 2014, Br J Clin Pharmacol 78 (3):572-86; Dahmane Elyes Ben Ali. 2013, Doctoral, Section des sciences pharmaceutiques, Université de Genève). Enzyme kinetics: (Desta Z. et al. 2004, J Pharmacol Exp Ther 310 (3):1062-75; Dickschen K. S. et al. 2012, Springer D. et al. 2003, Drug Metab Dispos 31 (8):979-82; Abdel-Rahman S. M. et al. 1999, Drug Metab Dispos 27 (7):770-5; Coller J. K. et al. 2002, Br J Clin Pharmacol 54 (2):157-67; Hu, X. X. et al. 2016, J Pharm Pharmacol 68 (6):819-25; Muroi, Y. et al. 2014, Drug Metab Pharmacokinet 29 (5):360-6). Enzyme regulation: (Zhao, X. J. et al. 2002, Xenobiotica 32 (10):863-78)

The population PK models include information about the concentration of drug and metabolites, transfer of drug between compartments, and clearance from the blood and liver. The concentration of tamoxifen in the brain has been studied, and this data was used to set these values (Tamoxifen in the Brain). The transfer of tamoxifen and endoxifen from the blood to the brain was calculated based on the known drug PK and the concentrations of the drugs in the tissues.

Tamoxifen metabolism is based on kinetic data and supported by PK data. The kinetic data were used to build and quantify tamoxifen metabolism. The PK data for tamoxifen metabolism is quantitatively similar to the kinetic data.

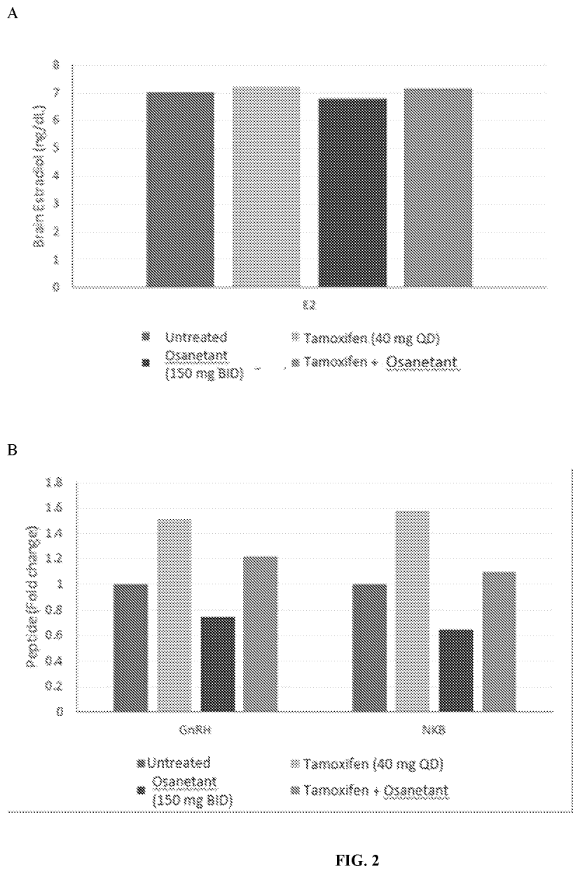

FIG. 2A demonstrates that in a post-menopausal virtual patient, treatment with tamoxifen may not cause large increases in estradiol. FIG. 2B demonstrates that in a post-menopausal virtual patient, treatment with tamoxifen may result in changes in GnRH and NKB.

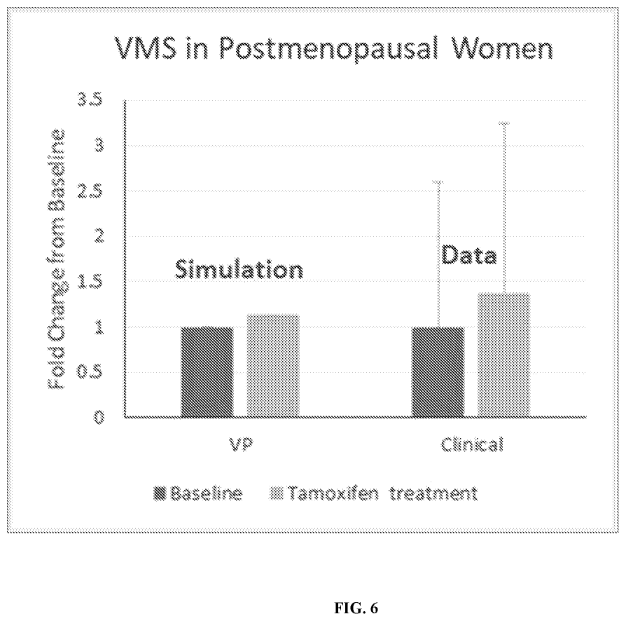

Results showing a comparison of effect of tamoxifen therapy in simulations vs. clinical data are shown in FIG. 6.

### Leuprolide

In the platform, leuprolide competes with GnRH for binding to the GnRH receptor. Over time, the higher leuprolide and faster degradation result in the loss of GnRH receptors, as would be seen in the pituitary. The receptor down-regulation will result in lower estrogen and testosterone production in the Platform. The decrease in sex hormone concentration will reduce the amount of hormone bound to the hormone receptor, triggering an increase in VMS.

A standard dose of leuprolide is 7.5 mg IM (monthly). Leuprolide PK is incorporated in the Platform as a 2-compartment model modified from (Lim, C. N. et al. 2015, Clin Pharmacokinet 54 (9):963-73). The Platform includes the 3 depots necessary to fit the complex dynamics of leuprolide pharmacokinetics. Additional references were considered in the development of the leuprolide PK and PD (Snelder N. et al. 2019, J Clin Pharmacol 85 (6):1247-1259; Lee D. S. et al. 2018, Molecules 23 (4); 909; Castellon E. et al. 2006, Cancer Invest 24 (3):261-8.).

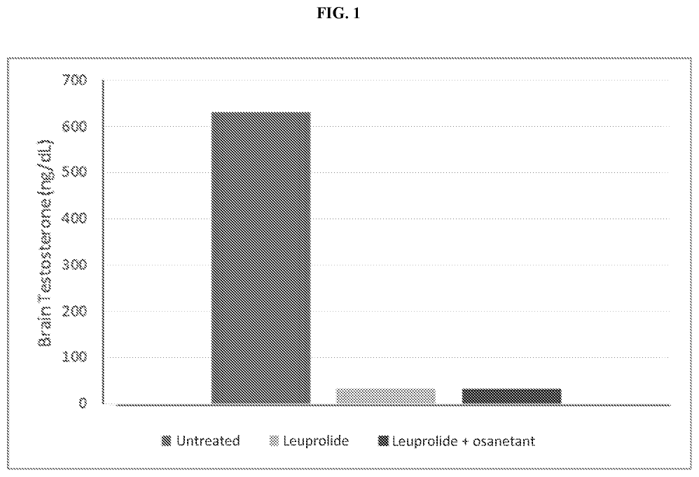

FIG. 1 demonstrates that in a male virtual patient, leuprolide treatment decreases testosterone to castration levels of <50 ng/dL. Osanetant co-administration does not increase the testosterone concentrations.

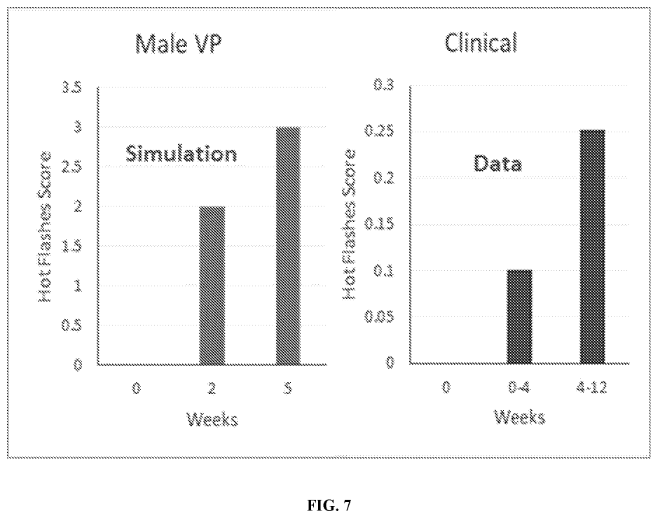

Results showing a comparison of effect of leuprolide therapy on VMS in simulations vs. clinical data are shown in FIG. 7.

### Osanetant

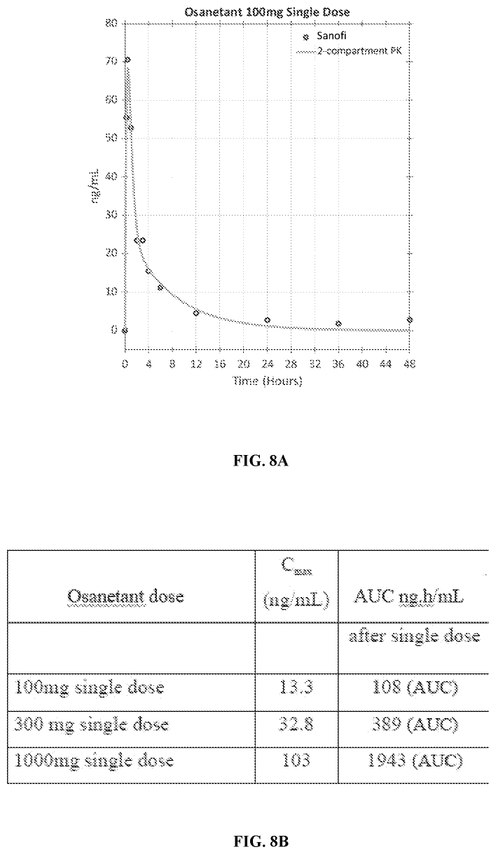

Osanetant (SR 142801) is an NK3 receptor antagonist originally identified by Sanofi. Patacchini R. et al. 1995, Eur J Pharmacol 278 (1):17-25; Nguyen-Le X. K. et al. 1996, Pharmacology 52 (5):283-91; Beaujouan J. C. et al. 1997, Eur J Pharmacol 319 (2-3):307-16; Emonds-Alt X. et al. 1995, Life Sci 56 (1):P127-32). Osanetant pharmacokinetics (PK) was derived from a Study Report by Sanofi describing concentration curves and PK summary data. A two-compartment PK model was derived from the data the concentration: time curves. FIG. 8 shows a comparison of the platform results with the clinical data. A two-compartment pharmacokinetic model was fit to the single-dose study in the Sanofi Study report.

Parameters
ka=0.039 min-1
Vol_periph=1768 L
Vol_central=620 L
Q=13.9 L*min-1
Blood_Liver_transport_k=0.011 min-1

Osanetant pharmacokinetics were also developed for a multiple-dose situation based on data from Sanofi. A variety of data were used to constrain osanetant pharmacokinetics.
Data from day 9 concentration/time curves used to fit PK parameters
Plasma compartment volume
Liver metabolism of osanetant (Emax)
The absorption rate constant for oral dosing
Bioavailability set to 40%
From osanetant Investigators Brochure
Liver volume set within the physiological range, same as tamoxifen
Transfer of drug between the liver and plasma constrained by
Tamoxifen values
Known liver blood flow
Liver concentration; Plasma concentration
Sanofi animal study data:
Brain/plasma concentration=0.8
Data from Malherbe P. et al. 2011, Expert Opin Ther Pat 21 (5):637-55; Iusuf D. et al. 2011, J Pharmacol Exp Ther 337 (3):710-7.

### Drug Interactions

There are two major pathways for the metabolism and clearance of tamoxifen: 4-hydroxylation (4-OH) and N-demethylation. The enzyme CYP2D6 modifies tamoxifen to the active metabolite 4-OH-tamoxifen. The production of 4-OH-tamoxifen by CYP2D6 is a small (7%) percentage of the overall metabolism of tamoxifen. Genetic variation in the CYP2D6 gene is associated with variation in plasma concentrations of N-desmethyl-4-hydroxytamoxifen (endoxifen) and is thought to account for up to approximately 50% of the variability in endoxifen concentrations (Dean 2012, “Tamoxifen Therapy and CYP2D6 Genotype.” In Medical Genetics Summaries, Bethesda (Md.): National Center for Biotechnology Information (US).). This variation in CYP2D6 activity may alter the clinical outcome for tamoxifen treatment.

Tamoxifen is metabolized by CYP3A4, with approximately 92% of the drug metabolized by this enzyme to form N-desmethyltamoxifen (Steams V. et al. 2003, J Natl Cancer Inst 95 (23):1758-64; Desta Z. et al. 2004, J Pharmacol Exp Ther 310 (3):1062-75; Kiyotani K. et al. 2012, Drug Metab Pharmacokinet 27 (1):122-31; Boocock D. J. et al. 2002, Carcinogenesis 23 (11):1897-901).

CYP3A4 metabolism in the liver is the primary clearance mechanism of osanetant (Sanofi-Synthelabo. 2003. Clinical Investigator's Brochure SR142801 Osanetant. Chilly-Mazarin France) and therefore, competition for the enzyme may occur. CYP3A4 is explicitly represented in the Platform to test the effect of co-administering tamoxifen and osanetant on the metabolism and plasma concentrations of the drugs. Osanetant also inhibits CYP2D6 activity (Sanofi-Synthelabo. 2003. Clinical Investigator's Brochure SR142801 Osanetant. Chilly-Mazarin France).

In the brain compartment of the Platform, osanetant competes with NKB for binding to the NK3 receptor. In the liver compartment of the Platform, osanetant competes with tamoxifen for CYP3A4 enzyme activity. Hepatic CYP2D6 activity is also included in the Platform to allow for testing of the impact of osanetant dosing on tamoxifen metabolism.

Steady-state liver tamoxifen, metabolite concentrations are predicted to be higher than osanetant. Platform simulations showed that when Tamoxifen is dosed at 40 mg QD, the steady-state hepatic concentrations of tamoxifen are: 0.5-3.8 uM, 40H tamoxifen: 0.06 uM, NDM tamoxifen: 7 uM, endoxifen: 24 uM. Osanetant CYP3A4 EC50 (2.5 uM) is estimated based on tamoxifen (2.5-8 uM) and midazolam (6 uM) EC50.

Leuprolide clearance occurs in the liver; however, because leuprolide is a peptide, its metabolism is not expected to compete with osanetant. Known leuprolide drug interactions are listed at https://www.drugs.com/drug-interactions/leuprolide,lupron-depot-index.html.

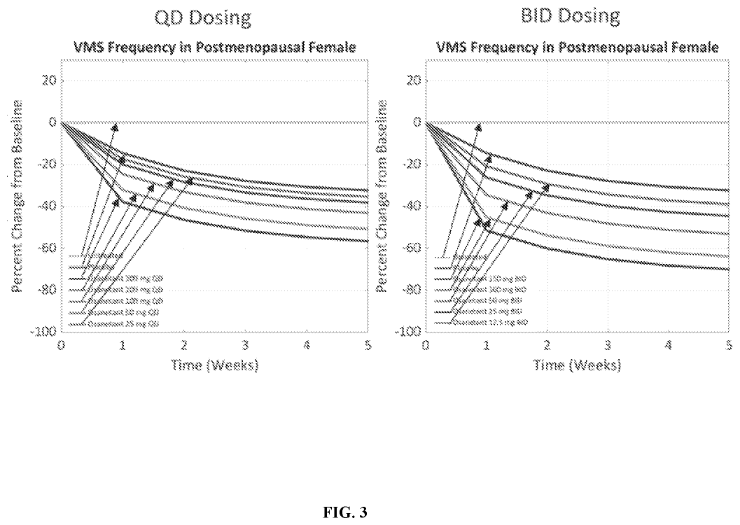

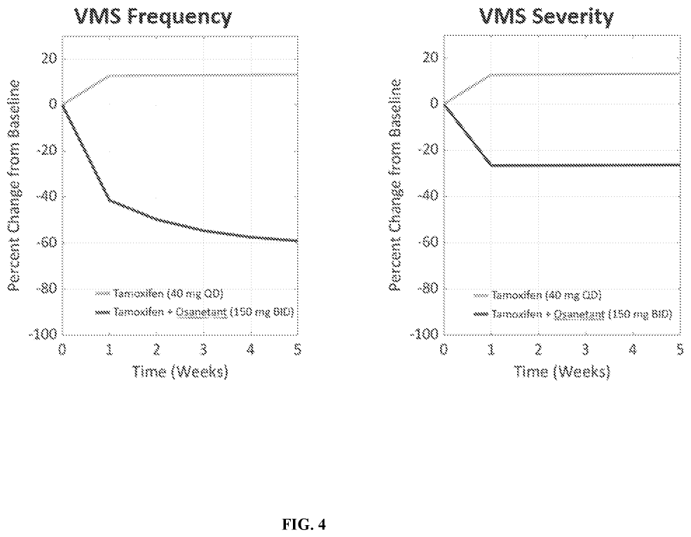

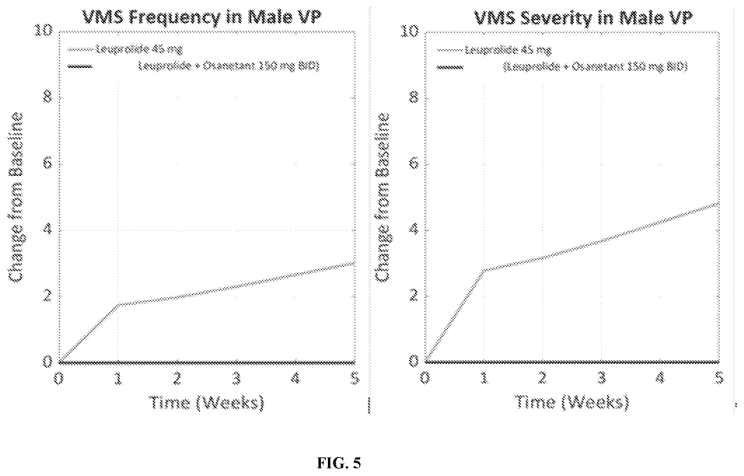

Research using the HPG QSP model described above demonstrates that osanetant is predicted to be efficacious in reducing NKB binding and vasomotor symptoms due to menopause or tamoxifen- or leuprolide-induced hormone deprivation. See FIGS. 3-5, evidencing twice daily dosing was superior in lowering VMS compared to once daily dosing.

### Example 4—Clinical Study of VMS in Bilateral Salpingo-Oophorectomy Patients

A Phase 2b, randomized, double-blind, placebo-controlled study to evaluate the efficacy and safety of osanetant on the prevention and treatment of moderate to severe vasomotor symptoms (VMS) in women who are undergoing risk-reducing bilateral salpingo-oophorectomy will be conducted.

Subjects will enter a 28-day screening period to determine study eligibility. Eligible subjects will be admitted to a clinical unit on Day −1 and will be randomized 1:1:1 to receive low (e.g., 25 mg, 50 mg, 100 mg, 200 mg, 300 mg or 400 mg once per day (QD)) or high doses of osanetant (e.g., 25 mg, 50 mg, 100 mg, 200 mg, 300 mg, or 400 mg, twice per day (BID)) or placebo. Subjects will be managed on an outpatient basis, with baseline (Day 1 and Day 4) collection of laboratory/pharmacokinetic (PK) assessments, will be dosed with osanetant on Day 1, will undergo bilateral salpingo-oophorectomy on Day 2, and undergo post-operative observation with basic laboratory assessment on Day 3. Subjects will continue to take study drug as an outpatient. Subjects will return to the clinic on Day 28 for study assessments and PK sampling. On Day 56, subjects will return to the clinic for study assessment and PK sampling and each dosing group will be re-randomized as follows: Placebo Group: Randomized 1:1:1 to receive low or high doses of osanetant or placebo. Low Dose Group: Randomized 1:1 to receive low dose of osanetant or placebo. High Dose Group: Randomized 1:1 to receive high dose of osanetant or placebo.

Subjects will continue their treatment and return to the clinic on Day 84 for study assessment, administration of last study drug, and PK sampling. Subjects will return on Day 112 for follow up assessments. Subjects are women who will be undergoing bilateral salpingo-oophorectomy.

40-60 subjects will be enrolled per treatment arm. Osanetant capsules will be administered orally.

Primary Objective: To evaluate the efficacy of osanetant compared to placebo on the change in frequency and severity of moderate to severe VMS in women who are undergoing bilateral salpingo-oophorectomy. Patient diary entries throughout the study will be maintained by the subjects. To evaluate the safety of osanetant in women undergoing bilateral salpingo-oophorectomy. Physical Examinations will cover Change from Baseline in vital signs; Analysis for arrhythmias by continuous Holter monitoring from Day −1 to Day 8 and Day 14 (24 hours); Abnormalities on 12-lead ECG; Change from Baseline in clinical laboratory assessments including lipids and coagulation profile; Change from Baseline in hormones including estradiol, testosterone, follicle stimulating hormone (FSH), adrenocorticotropic hormone (ACTH), cortisol, thyroid-stimulating hormone (TSH), triiodothyronine (T3) and thyroxine (T4); Adverse Events (AE).

Secondary Objective: To evaluate the effect of osanetant compared to placebo on the change in frequency and severity of mild, moderate, and severe VMS in women undergoing bilateral salpingo-oophorectomy at day 14, 28 and 56. To evaluate the effect of osanetant compared to placebo on quality of life (QOL) related to vasomotor symptoms in women undergoing bilateral salpingo-oophorectomy. To assess the effects of osanetant compared to placebo on hot flash frequency and severity of nighttime awakenings (NTA). To evaluate the PK of osanetant. Patient diary entries and questionnaires will be used, e.g., Hot Flash Related Daily Interference Scale (HFRDIS), Pittsburgh Sleep Quality Index (PSQI), Insomnia Severity Index (ISI), Menopause Specific Quality of Life (MENQoL) questionnaires measure change from baseline.

Exploratory Objective: To evaluate the efficacy (treatment) of osanetant after development of VMS in women who are undergoing bilateral salpingo-oophorectomy relative to baseline on the frequency and severity of moderate to severe vasomotor symptoms at Day 56 and Day 84. To evaluate the efficacy of osanetant after discontinuation in women who are undergoing bilateral salpingo-oophorectomy relative to baseline on the frequency and severity of moderate to severe vasomotor symptoms. To evaluate the effect of osanetant compared to placebo on the change in pharmacodynamic markers in women undergoing bilateral salpingo-oophorectomy. To assess PK exposure-response relationships between osanetant for efficacy and safety endpoints. To assess the impact of osanetant on stress hormones. Patient diary entries and plasma/blood sample measurements will be used.

Inclusion Criteria: Female subjects over the age of 18 who are undergoing bilateral salpingo-oophorectomy. Able to understand and comply with the requirements of the study and sign Informed Consent forms.

Exclusion Criteria: Any active comorbid disease deemed by the investigator to be clinically significant which could impact safety during study conduct including renal or hepatic impairment. Serum creatinine laboratory value greater than 1.2 times upper limit of normal (ULN) reference range (after adjustment for age) at Screening or Day −1 or subjects with renal function glomerular filtration rate (GFR)<60 mL/min/1.73 m2 based on the modification of diet in renal disease (MDRD) equation. Total bilirubin greater than upper limit of normal reference range (with the exception of Gilbert's Syndrome) and/or alanine transaminase (ALT) >2 times ULN reference ranges and/or aspartate amino transferase (AST) >2 times ULN reference ranges at Screening or Day −1. Any active medicinal (pharmaceutical or homeopathic) therapy considered by the investigator to potentially cause hepatic impairment leading to elevated alanine transaminase (ALT) ranges and/or aspartate amino transferase (AST) ranges. Active or ongoing health conditions that could cause difficulty in interpretation of hot flashes. Subjects with a prior medical history of or an increased risk of seizures, or who have a history of recent (within 6 months of Screening) head trauma that resulted in a loss of consciousness or concussion. Any prior or ongoing history of arrhythmias. Any ongoing cardiovascular disease including heart failure, coronary artery disease, or uncontrolled hypertension or uncontrolled diabetes. Any clinically relevant ECG abnormalities at Screening. Use of any prohibited medications strong or moderate such as cytochrome P450 (CYP) 3A4 inhibitors, hormone replacement therapy (HRT), hormonal contraceptive, any treatment for VMS [prescription, over the counter or herbal]) or subject not willing to wash out and discontinue such drugs for the full extent of the study. Any active ongoing condition that could cause difficulty in interpreting vasomotor symptoms such as: infection that could cause pyrexia, pheochromocytoma, hyperthyroidism, carcinoid syndrome, alcohol abuse. Body mass index (BMI) >38. Inability to complete questionnaires or patient diary for any reason including psychiatric disorders and inability or unwillingness to use electronic devices. History of hypothalamic dysfunction. Any clinically significant or unstable medical or psychiatric condition that would interfere with the subject's ability to participate in the study including anxiety syndromes. Any clinically significant abnormal laboratory test result(s) measured at Screening or on Day −1. Subjects who, in the opinion of the Investigator, should not participate in the study for any other reason. Any known allergy or hypersensitivity to any of the ingredients in the study medication or to skin adhesives. Current or planned use of other agents for treating hot flashes Participation in another investigational drug, biologic, or medical device trial within 30 days prior to Screening.

Analysis Populations: The intention to treat (ITT) population will be used for the analyses of the primary and secondary endpoints. Additional supportive efficacy and sensitivity analyses will be based on the per-protocol who did not have any major protocol violations. The Safety population will be used for the analyses of AE(s) and includes all randomized subjects receiving at least 1 dose of study treatment. The PK Analysis population will include all subjects who provide PK samples.

Safety and Efficacy Analysis: Safety, and efficacy data will be presented by means of descriptive statistics and figures, as appropriate, by treatment. All AEs will be coded by the Sponsor (or designee) using the current version of the Medical Dictionary for Regulatory Activities to assign system organ class and preferred term classification to events and diseases, based on the original terms entered on the electronic case report form. The incidence of AEs will be summarized by system organ class, preferred term, relationship to study treatment, and severity for each study treatment. All AEs, including AEs that lead to premature discontinuation from the study and from study treatment and serious AEs (SAEs), will be recorded.

All other safety measures including laboratory tests, vital signs, concomitant medication, medical history, physical examination, and ECG data will also be summarized descriptively (mean, standard deviation, median, minimum and maximum). Laboratory tests will also be summarized by absolute and percent change from baseline and listed by significant values. Subjects to record number of times awoken during the night due to flashes (Night-time awakening [NTA]). Subjects to record the occurrence and severity of the flash (scale 1 to 3) in a continuous diary. Study medication will be dispensed to the subjects on Days 1, 28, and 56.

### Example 5—Clinical Study of VMS in HR(+) Breast Cancer Patients

A Phase 2b, randomized, double-blind, placebo-controlled study to evaluate the efficacy and safety of osanetant on the prevention and treatment of moderate to severe vasomotor symptoms (VMS) in women with hormone receptor positive (HR(+)) breast cancer and receiving tamoxifen therapy will be conducted.

Subjects will enter a 28-day screening period to determine study eligibility. Eligible subjects will be admitted to a clinical unit on Day −1 and will be randomized 1:1:1 to receive low (e.g., 25 mg, 50 mg, 100 mg, 200 mg, 300 mg or 400 mg once per day (QD)) or high doses of osanetant (e.g., 25 mg, 50 mg, 100 mg, 200 mg, 300 mg or 400 mg twice per day (BID)) or placebo. Subjects will be managed on an outpatient basis, with baseline (Day 1 and Day 4) collection of laboratory/pharmacokinetic (PK) assessments, will be dosed with osanetant on Day 1, will initiate tamoxifen therapy on Day 2, and come in for basic laboratory assessment on Day 3. Subjects will continue to take study drug as an outpatient. Subjects will return to the clinic on Day 28 for study assessments and PK sampling. On Day 56, subjects will return to the clinic for study assessment and PK sampling and each dosing group will be re-randomized as follows: Placebo Group: Randomized 1:1:1 to receive low or high doses of osanetant or placebo. Low Dose Group: Randomized 1:1 to receive low dose of osanetant or placebo. High Dose Group: Randomized 1:1 to receive high dose of osanetant or placebo.

Subjects will continue their treatment and return to the clinic on Day 84 for study assessment, administration of last study drug, and PK sampling. Subject will return on Day 112 for follow up assessments. Subjects are women with HR(+) breast cancer who will be initiating tamoxifen therapy.

40-60 subjects will be enrolled per treatment arm. Osanetant capsules will be administered orally. Study drug will be administered in the morning from Day 1 to Day 3. Study medication will be dispensed to the subjects on Days 4, 14, 28, and 84.

Primary Objective: To evaluate the efficacy of osanetant in women with HR(+) breast cancer who will be initiating tamoxifen therapy relative to baseline on the frequency and severity of moderate to severe VMS. Patient diary entries throughout the study. To evaluate the safety of osanetant in women with HR(+) breast cancer who will be initiating tamoxifen therapy. Physical Examinations will cover Change from Baseline in vital signs; Analysis for arrhythmias by continuous Holter monitoring from Day −1 to Day 8 and Day 14 (24 hours); Abnormalities on 12-lead ECG; Change from Baseline in clinical laboratory assessments including lipids and coagulation profile; Change from Baseline in hormones including estradiol, testosterone, follicle stimulating hormone (FSH), adrenocorticotropic hormone (ACTH), cortisol, thyroid-stimulating hormone (TSH), triiodothyronine (T3) and thyroxine (T4); Adverse Events (AE).

Secondary Objective: To evaluate the effect of osanetant compared to placebo on the change in frequency and severity of mild, moderate, and severe VMS in women with HR(+) breast cancer who will be initiating tamoxifen therapy. To evaluate the effect of osanetant compared to placebo on quality of life (QOL) related to vasomotor symptoms in women with HR(+) breast cancer who will be initiating tamoxifen therapy. To assess the effects of osanetant compared to placebo on hot flash frequency and severity of nighttime awakenings (NTA). To evaluate the PK of osanetant. Patient diary entries and questionnaires will be used, e.g., Pittsburgh Sleep Quality Index (PSQI), Insomnia Severity Index (ISI), Menopause Specific Quality of Life (MENQoL) questionnaires measure change from baseline.

Exploratory Objective: To evaluate the efficacy (treatment) of osanetant after development of VMS in women with HR(+) breast cancer who will be initiating tamoxifen therapy relative to baseline on the frequency and severity of moderate to severe vasomotor symptoms. To evaluate the efficacy of osanetant after discontinuation in women with HR(+) breast cancer who will be initiating tamoxifen therapy relative to baseline on the frequency and severity of moderate to severe vasomotor symptoms. To evaluate the effect of osanetant compared to placebo on the change in pharmacodynamic markers in women with HR(+) breast cancer who will be initiating tamoxifen therapy. To assess PK exposure-response relationships between osanetant for efficacy and safety endpoints. To assess the impact of osanetant on stress hormones. Patient diary entries and plasma/blood sample measurements will be used.

Inclusion Criteria: Female subjects over the age of 18 who have HR(+) breast cancer and who will be initiating tamoxifen therapy. Able to understand and comply with the requirements of the study and sign Informed Consent forms.

Exclusion Criteria: Any active comorbid disease, other than (HR+) breast cancer, deemed by the investigator to be clinically significant which could impact safety during study conduct including renal or hepatic impairment. Serum creatinine laboratory value greater than 1.2 times upper limit of normal (ULN) reference range (after adjustment for age) at Screening or Day −1 or subjects with renal function glomerular filtration rate (GFR)<60 mL/min/1.73 m² based on the modification of diet in renal disease (MDRD) equation. Total bilirubin greater than upper limit of normal reference range (with the exception of Gilbert's Syndrome) and/or alanine transaminase (ALT) >2 times ULN reference ranges and/or aspartate amino transferase (AST) >2 times ULN reference ranges at Screening or Day −1. Any active medicinal (pharmaceutical or homeopathic) therapy considered by the investigator to potentially cause hepatic impairment leading to elevated alanine transaminase (ALT) ranges and/or aspartate amino transferase (AST) ranges. Active or ongoing health conditions that could cause difficulty in interpretation of hot flashes. Subjects with a prior medical history of or an increased risk of seizures, or who have a history of recent (within 6 months of Screening) head trauma that resulted in a loss of consciousness or concussion. Any prior or ongoing history of arrhythmias. Any ongoing cardiovascular disease including heart failure, coronary artery disease, or uncontrolled hypertension or uncontrolled diabetes. Any clinically relevant ECG abnormalities at Screening. Use of any prohibited medications strong or moderate such as cytochrome P450 [CYP] 3A4 inhibitors, hormone replacement therapy [HRT], hormonal contraceptive, any treatment for VMS [prescription, over the counter or herbal]) or subject not willing to wash out and discontinue such drugs for the full extent of the study. Any active ongoing condition that could cause difficulty in interpreting vasomotor symptoms such as: infection that could cause pyrexia, pheochromocytoma, hyperthyroidism, carcinoid syndrome, alcohol abuse. Body mass index (BMI) >38. Inability to complete questionnaires or patient diary for any reason including psychiatric disorders and inability or unwillingness to use electronic devices. History of hypothalamic dysfunction. Any clinically significant or unstable medical or psychiatric condition that would interfere with the subject's ability to participate in the study including anxiety syndromes. Any clinically significant abnormal laboratory test result(s) measured at Screening or on Day −1. Subjects who, in the opinion of the Investigator, should not participate in the study for any other reason. Any known allergy or hypersensitivity to any of the ingredients in the study medication or to skin adhesives. Current or planned use of other agents for treating hot flashes Participation in another investigational drug, biologic, or medical device trial within 30 days prior to Screening.

Analysis Populations: The intention to treat (ITT) population will be used for the analyses of the primary and secondary endpoints. Additional supportive efficacy and sensitivity analyses will be based on the per-protocol who did not have any major protocol violations. The Safety population will be used for the analyses of AE(s) and includes all randomized subjects receiving at least 1 dose of study treatment. The PK Analysis population will include all subjects who provide PK samples.

Safety and Efficacy Analysis: Safety, and efficacy data will be presented by means of descriptive statistics and figures, as appropriate, by treatment. All AEs will be coded by the Sponsor (or designee) using the current version of the Medical Dictionary for Regulatory Activities to assign system organ class and preferred term classification to events and diseases, based on the original terms entered on the electronic case report form. The incidence of AEs will be summarized by system organ class, preferred term, relationship to study treatment, and severity for each study treatment. All AEs, including AEs that lead to premature discontinuation from the study and from study treatment and serious AEs (SAEs), will be recorded.

All other safety measures including laboratory tests, vital signs, concomitant medication, medical history, physical examination, and ECG data will also be summarized descriptively (mean, standard deviation, median, minimum and maximum). Laboratory tests will also be summarized by absolute and percent change from baseline and listed by significant values. Subjects to record number of times awoken during the night due to flashes (Night-time awakening [NTA]). Subjects to record the occurrence and severity of the flash (scale 1 to 3) in a continuous diary. Luteinizing hormone (LH) sampling every 60 mins from time 0 (pre-dose on Day 1 and Day 7) to 8 hrs. The same sampling schedule is required on Day −1, starting at a similar time to that planned for Days 1 and 7. PK Sampling at Pre-dose; 0.5; 1.0; 1.5; 2.0; 2.5; 3.0; 4.0; 5.0; 6.0; 8.0; and 12.0 hours. Additional 24-hour PK sample will be taken on Day 56 (approximately 30 min prior to Day 57 dose). Pre-dose samples for PK and LH to be taken within 30 minutes prior to dose administration.

### Example 6—Clinical Study of VMS in HR(+) Prostate Cancer Patients

A Phase 2b, randomized, double-blind, placebo-controlled study to evaluate the efficacy and safety of osanetant on the prevention and treatment of moderate to severe vasomotor symptoms (VMS) in men with hormone receptor positive (HR(+)) prostate cancer and receiving leuprolide therapy will be conducted.

Subjects will enter a 28-day screening period to determine study eligibility. Eligible subjects will be admitted to a clinical unit on Day −1 and will be randomized 1:1:1 to receive low (e.g., 25 mg, 50 mg, 100 mg, 200 mg, 300 mg or 400 mg once per day (QD)) or high doses of osanetant (e.g., 25 mg, 50 mg, 100 mg, 200 mg, 300 mg or 400 mg twice per day (BID)) or placebo. Subjects will be managed on an outpatient basis, with baseline (Day 1 and Day 4) collection of laboratory/pharmacokinetic (PK) assessments, will be dosed with osanetant on Day 1, will initiate leuprolide therapy on Day 2, and come in for basic laboratory assessment on Day 3. Subjects will continue to take study drug as an outpatient. Subjects will return to the clinic on Day 14 and Day 28 for study assessments and PK sampling. On Day 56, subjects will return to the clinic for study assessment and PK sampling and each dosing group will be re-randomized as follows: Placebo Group: Randomized 1:1:1 to receive low or high doses of osanetant or placebo. Low Dose Group: Randomized 1:1 to receive low dose of osanetant or placebo. High Dose Group: Randomized 1:1 to receive high dose of osanetant or placebo.

Subjects will continue their treatment and return to the clinic on Day 84 for study assessment, administration of last study drug, and PK sampling. Subject will return on Day 112 for follow up assessments. Subjects are men with HR(+) prostate cancer who will be initiating leuprolide therapy.

40-60 subjects will be enrolled per treatment arm. Osanetant capsules will be administered orally. Study drug will be administered in the morning from Day 1 to Day 3. Study medication will be dispensed to the subjects on Days 4, 14, 28, and 84.

Primary Objective: To evaluate the efficacy of osanetant in men with HR(+) prostate cancer who will be initiating leuprolide therapy relative to baseline on the frequency and severity of moderate to severe VMS. Patient diary entries throughout the study. To evaluate the safety of osanetant in men with HR(+) prostate cancer who will be initiating leuprolide therapy. Physical Examinations will cover Change from Baseline in vital signs; Analysis for arrhythmias by continuous Holter monitoring from Day −1 to Day 8 and Day 14 (24 hours); Abnormalities on 12-lead ECG; Change from Baseline in clinical laboratory assessments including lipids and coagulation profile; Change from Baseline in hormones including estradiol, testosterone, follicle stimulating hormone (FSH), adrenocorticotropic hormone (ACTH), cortisol, thyroid-stimulating hormone (TSH), triiodothyronine (T3) and thyroxine (T4); Adverse Events (AE).

Secondary Objective: To evaluate the effect of osanetant compared to placebo on the change in frequency and severity of mild, moderate, and severe VMS in men with HR(+) prostate cancer who will be initiating leuprolide therapy. To evaluate the effect of osanetant compared to placebo on quality of life (QOL) related to vasomotor symptoms in men with HR(+) prostate cancer who will be initiating leuprolide therapy. To assess the effects of osanetant compared to placebo on hot flash frequency and severity of nighttime awakenings (NTA). To evaluate the PK of osanetant. Patient diary entries and questionnaires will be used where applicable.

Exploratory Objective: To evaluate the efficacy (treatment) of osanetant after development of VMS in men with HR(+) prostate cancer who will be initiating leuprolide therapy relative to baseline on the frequency and severity of moderate to severe vasomotor symptoms. To evaluate the efficacy of osanetant after discontinuation in men with HR(+) prostate cancer who will be initiating leuprolide therapy relative to baseline on the frequency and severity of moderate to severe vasomotor symptoms. To evaluate the effect of osanetant compared to placebo on the change in pharmacodynamic markers in men with HR(+) prostate cancer who will be initiating leuprolide therapy. To assess PK exposure-response relationships between osanetant for efficacy and safety endpoints. To assess the impact of osanetant on stress hormones. Patient diary entries and plasma/blood sample measurements will be used.

Inclusion Criteria: Male subjects over the age of 18 who have HR(+) prostate cancer and who will be initiating leuprolide therapy. Able to understand and comply with the requirements of the study and sign Informed Consent forms.

Exclusion Criteria: Any active comorbid disease, other than (HR+) prostate cancer, deemed by the investigator to be clinically significant which could impact safety during study conduct including renal or hepatic impairment. Serum creatinine laboratory value greater than 1.2 times upper limit of normal (ULN) reference range (after adjustment for age) at Screening or Day −1 or subjects with renal function glomerular filtration rate (GFR)<60 mL/min/1.73 m² based on the modification of diet in renal disease (MDRD) equation. Total bilirubin greater than upper limit of normal reference range (with the exception of Gilbert's Syndrome) and/or alanine transaminase (ALT) >2 times ULN reference ranges and/or aspartate amino transferase (AST) >2 times ULN reference ranges at Screening or Day −1. Any active medicinal (pharmaceutical or homeopathic) therapy considered by the investigator to potentially cause hepatic impairment leading to elevated alanine transaminase (ALT) ranges and/or aspartate amino transferase (AST) ranges. Active or ongoing health conditions that could cause difficulty in interpretation of hot flashes. Subjects with a prior medical history of or an increased risk of seizures, or who have a history of recent (within 6 months of Screening) head trauma that resulted in a loss of consciousness or concussion. Any prior or ongoing history of arrhythmias. Any ongoing cardiovascular disease including heart failure, coronary artery disease, or uncontrolled hypertension or uncontrolled diabetes. Any clinically relevant ECG abnormalities at Screening. Use of any prohibited medications strong or moderate such as cytochrome P450 [CYP]3A4 inhibitors, hormone replacement therapy [HRT], hormonal contraceptive, any treatment for VMS [prescription, over the counter or herbal]) or subject not willing to wash out and discontinue such drugs for the full extent of the study. Any active ongoing condition that could cause difficulty in interpreting vasomotor symptoms such as: infection that could cause pyrexia, pheochromocytoma, hyperthyroidism, carcinoid syndrome, alcohol abuse. Body mass index (BMI) >38. Inability to complete questionnaires or patient diary for any reason including psychiatric disorders and inability or unwillingness to use electronic devices. History of hypothalamic dysfunction. Any clinically significant or unstable medical or psychiatric condition that would interfere with the subject's ability to participate in the study including anxiety syndromes. Any clinically significant abnormal laboratory test result(s) measured at Screening or on Day −1. Participation in any clinical research study evaluating another investigational drug or therapy within 30 days or within 5 half-lives (whichever is longer), of the investigational drug prior to consenting to study entry. If the subject is in an observational clinical study no washout is required. Subjects who, in the opinion of the Investigator, should not participate in the study for any other reason. Any known allergy or hypersensitivity to any of the ingredients in the study medication or to skin adhesives. Current or planned use of other agents for treating hot flashes

Analysis Populations: The intention to treat (ITT) population will be used for the analyses of the primary and secondary endpoints. Additional supportive efficacy and sensitivity analyses will be based on the per-protocol who did not have any major protocol violations. The Safety population will be used for the analyses of AE(s) and includes all randomized subjects receiving at least 1 dose of study treatment. The PK Analysis population will include all subjects who provide PK samples.

Safety and Efficacy Analysis: Safety, and efficacy data will be presented by means of descriptive statistics and figures, as appropriate, by treatment. All AEs will be coded by the Sponsor (or designee) using the current version of the Medical Dictionary for Regulatory Activities to assign system organ class and preferred term classification to events and diseases, based on the original terms entered on the electronic case report form. The incidence of AEs will be summarized by system organ class, preferred term, relationship to study treatment, and severity for each study treatment. All AEs, including AEs that lead to premature discontinuation from the study and from study treatment and serious AEs (SAEs), will be recorded.

All other safety measures including laboratory tests, vital signs, concomitant medication, medical history, physical examination, and ECG data will also be summarized descriptively (mean, standard deviation, median, minimum and maximum). Laboratory tests will also be summarized by absolute and percent change from baseline and listed by significant values. Subjects to record number of times awoken during the night due to flashes (Night-time awakening [NTA]). Subjects to record the occurrence and severity of the flash (scale 1 to 3) in a continuous diary. Luteinizing hormone (LH) sampling every 60 mins from time 0 (pre-dose on Day 1 and Day 7) to 8 hrs. The same sampling schedule is required on Day −1, starting at a similar time to that planned for Days 1 and 7. PK Sampling at Pre-dose; 0.5; 1.0; 1.5; 2.0; 2.5; 3.0; 4.0; 5.0; 6.0; 8.0; and 12.0 hours. Additional 24-hour PK sample taken on Day 56 (approximately 30 min prior to Day 57 dose). Pre-dose samples for PK and LH to be taken within 30 minutes prior to dose administration.

All other safety measures including laboratory tests, vital signs, concomitant medication, medical history, physical examination, and ECG data will also be summarized descriptively (mean, standard deviation, median, minimum and maximum). Laboratory tests will also be summarized by absolute and percent change from baseline and listed by significant values

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method of blocking, attenuating, or limiting the development of one or more vasomotor symptoms (VMS) in a patient who has cancer, has had cancer, or has an increased risk for cancer, wherein the patient will be undergoing hormone deprivation therapy, a medical and/or surgical procedure that may cause VMS, comprising administering an effective amount of a neurokinin receptor (NK) antagonist, for a time period prior to, and optionally concurrently with, the hormone deprivation therapy, a medical and/or surgical procedure.

2. The method of claim 1, wherein the neurokinin receptor antagonist is a neurokinin-3 receptor (NK3) antagonist.

3. The method of claim 2, wherein the neurokinin-3 receptor antagonist is selected from osanetant, fezolinetant, pavinetant, talnetant, (S)-3-methyl-2-phenyl-N-(1-phenylpropyl)-4-quinolinecarboxamide (SB-222,200), (−)-(R)—N-(α-methoxycarbonylbenzyl)-2-phenylquinoline-4-carboxamide (SB-218,795), 2-[3,5-bis(trifluoromethyl)phenyl]-N-{4-(4-fluoro-2-methylphenyl)-6-[(7S,9aS)-7-(hydroxymethyl)hexahydropyrazino[2,1-c] [1,4]oxazin-8(1H)-yl]pyridin-3-yl}-N,2-dimethylpropanamide (NT-814), or a stereoisomer, mixture of stereoisomers, prodrug, pharmaceutically acceptable salt, hydrate, solvate, acid salt hydrate, N-oxide or isomorphic crystalline form thereof.

4. The method of claim 3, wherein the neurokinin-3 receptor antagonist is osanetant or a stereoisomer, mixture of stereoisomers, prodrug, pharmaceutically acceptable salt, hydrate, solvate, acid salt hydrate, N-oxide or isomorphic crystalline form thereof.

5. The method of claim 4, wherein the effective amount of osanetant is less than about 400 mg per day.

6. The method of claim 5, wherein the effective amount of osanetant is from about 10 to about 350 mg per day.

7. The method of claim 5, wherein the effective amount of osanetant is less than about 200 mg per day

8. The method of claim 7, wherein the effective amount of osanetant is from about 10 to about 150 mg per day.

9. The method of claim 4, wherein the effective amount of osanetant is about 300 mg per day.

10. The method of claim 9, wherein the osanetant is administered once a day.

11. The method of claim 9, wherein the osanetant is administered twice a day, each dose being about 150 mg.

12. The method of any preceding claim, wherein hormone therapy for the patient is contraindicated.

13. The method of claim 12, wherein the hormone therapy is estrogen therapy.

14. The method of claim 1, wherein the hormone deprivation therapy is treatment with a selective estrogen receptor modulator (SERM).

15. The method of claim 14, wherein the SERM is tamoxifen.

16. The method of claim 14, wherein the patient is a female patient.

17. The method of claim 14, wherein the patient is a post-menopausal female patient.

18. The method of claim 1, wherein the hormone deprivation therapy is treatment with a gonadotropin-releasing hormone (GnRH) agonist or antagonist.

19. The method of claim 18, wherein the patient is a male patient.

20. The method of claim 18, wherein the GnRH agonist is leuprolide.

21. The method of claim 1, wherein the hormone deprivation therapy is treatment with a selective estrogen receptor degrader (SERD).

22. The method of any preceding claim, wherein the cancer is breast cancer, ovarian cancer, uterine cancer, or prostate cancer.

23. The method of any preceding claim, wherein the cancer is hormone receptor-positive cancer.

24. The method of any preceding claim, wherein the cancer is breast cancer.

25. The method of any one of claims 1-13, 18-20, or 22-23, wherein the cancer is prostate cancer.

26. The method of any proceeding claim, wherein the patient has tested positive for a BRCA1, BRCA2 or PALB2 mutation.

27. The method of any preceding claim, wherein the time period for administration of the NK antagonist prior to the hormone deprivation therapy, a medical and/or surgical procedure is about 12 weeks.

28. The method of any preceding claim, wherein the time period over which the NK antagonist is administered prior to the hormone deprivation therapy, a medical and/or surgical procedure is about 8 weeks.

29. The method of any preceding claim, wherein the time period over which the NK antagonist is administered prior to the hormone deprivation therapy, a medical and/or surgical procedure is about 4 weeks.

30. The method of any preceding claim, wherein the time period over which the NK antagonist is administered prior to the hormone deprivation therapy, a medical and/or surgical procedure is about one week.

31. The method of any preceding claim, wherein the patient continues to receive a NK antagonist after the hormone deprivation therapy, a medical and/or surgical procedure.

32. The method of any preceding claim, wherein the NK antagonist is administered concurrently with hormone deprivation therapy, a medical and/or surgical procedure.

33. The method of any one of the preceding claims, further comprising administering one or more of an additional therapeutic agent.

34. The method of claim 33, wherein the additional therapeutic agent is a selective estrogen receptor modulator (SERM).

35. The method of claim 33, wherein the SERM is tamoxifen.

36. The method of claim 33, wherein the additional therapeutic agent is a gonadotropin-releasing hormone (GnRH) agonist or antagonist.

37. The method of claim 33, wherein the GnRH agonist is leuprolide.

38. The method of claim 33, wherein the additional therapeutic agent is a nonsteroidal antiandrogen.

39. The method of claim 33, wherein the additional therapeutic agent is a kappa opioid agonist.

40. The method of claim 33, wherein the additional therapeutic agent is a SERD.

41. A method of preventing hypertrophy of kisspeptin/neurokinin B/dynorphin (KNDy) neurons in a patient in need thereof by administering to said patient an effective amount of a NK antagonist.

42. The method of claim 41, wherein the NK antagonist is osanetant or a stereoisomer, mixture of stereoisomers, prodrug, pharmaceutically acceptable salt, hydrate, solvate, acid salt hydrate, N-oxide or isomorphic crystalline form thereof.

43. A method for reducing the frequency and severity of hormone deprivation therapy-induced vasomotor symptoms or surgery-induced vasomotor symptoms in a cancer patient, the method comprising administering a combination of a hormone antagonist and an NK antagonist to the cancer patient in need thereof, wherein the NK antagonist is administered twice a day, each dose comprising from about 100 mg to about 200 mg of the NK antagonist.

44. The method of claim 43, wherein the NK antagonist is a NK3 antagonist.

45. The method of claim 44, wherein the NK3 antagonist is osanetant, or a pharmaceutically acceptable salt thereof.

46. The method of claim 43, wherein the cancer patient is a BRCA1/2 positive breast cancer patient.

47. The method of any one of claims 43-46, wherein the NK antagonist is administered twice a day, each dose comprising about 150 mg of the NK antagonist

48. A method for reducing the frequency and severity of tamoxifen-induced vasomotor symptoms or surgery-induced vasomotor symptoms in a cancer patient, the method comprising administering a combination of tamoxifen and an NK antagonist to the cancer patient in need thereof, wherein the NK antagonist is administered twice a day, each dose comprising from about 100 mg to about 200 mg of the NK antagonist.

49. The method of claim 48, wherein the NK antagonist is a NK3 antagonist.

50. The method of claim 49, wherein the NK3 antagonist is osanetant, or a pharmaceutically acceptable salt thereof.

51. The method of claim 48, wherein the cancer patient is a BRCA1/2 positive breast cancer patient.

52. The method of any one of claims 48-51, wherein the NK antagonist is administered twice a day, each dose comprising about 150 mg of the NK antagonist.

53. A method for reducing leuprolide-induced vasomotor symptoms or surgery-induced vasomotor symptoms in a cancer patient, the method comprising administering a combination of leuprolide and an NK antagonist to the cancer patient in need thereof, wherein the NK antagonist is administered twice a day, each dose comprising from about 100 mg to about 200 mg of the NK antagonist.

54. The method of claim 53, wherein the NK antagonist is a NK3 antagonist.

55. The method of claim 54, wherein the NK3 antagonist is osanetant, or a pharmaceutically acceptable salt thereof.

56. The method of claim 53, wherein the cancer patient is a prostate cancer patient.

57. The method of any one of claims 53-56, wherein the NK antagonist is administered twice a day, each dose comprising about 150 mg of the NK antagonist.

58. A method for reducing the frequency and severity of tamoxifen-induced vasomotor symptoms or surgery-induced vasomotor symptoms in a cancer patient, the method comprising administering a combination of tamoxifen and an NK antagonist to the cancer patient in need thereof, wherein the NK antagonist is administered twice a day, each dose comprising from about 25 mg to about 100 mg of the NK antagonist.

59. The method of claim 58, wherein the NK antagonist is a NK3 antagonist.

60. The method of claim 59, wherein the NK3 antagonist is osanetant, or a pharmaceutically acceptable salt thereof.

61. The method of claim 58, wherein the cancer patient is a BRCA1/2 positive breast cancer patient or a HR positive breast cancer patient.

62. The method of any one of claims 58-61, wherein the NK antagonist is administered twice a day, and the total daily dose of the NK antagonist ranges from about 50 mg per day to about 200 mg per day.

63. The method of any one of claims 58-62, wherein the NK antagonist is administered prior to initiation of tamoxifen treatment or prior to surgery for a period of less than one week.

64. A method for reducing leuprolide-induced vasomotor symptoms or surgery-induced vasomotor symptoms in a cancer patient, the method comprising administering a combination of leuprolide and an NK antagonist to the cancer patient in need thereof, wherein the NK antagonist is administered twice a day, each dose comprising from about 25 mg to about 100 mg of the NK antagonist.

65. The method of claim 64, wherein the NK antagonist is a NK3 antagonist.

66. The method of claim 65, wherein the NK3 antagonist is osanetant, or a pharmaceutically acceptable salt thereof.

67. The method of claim 64, wherein the cancer patient is a prostate cancer patient.

68. The method of any one of claims 64-67, wherein the NK antagonist is administered twice a day, and the total daily dose of the NK antagonist ranges from about 50 mg per day to about 200 mg per day.

69. The method of any one of claims 64-68, wherein the NK antagonist is administered prior to initiation of leuprolide treatment or prior to surgery for a period of less than one week.

70. A method for reducing the frequency and severity of vasomotor symptoms in a patient undergoing bilateral salpingo-oophorectomy, the method comprising administering an NK antagonist to the patient in need thereof, wherein the NK antagonist is administered twice a day, each dose comprising from about 25 mg to about 100 mg of the NK antagonist.

71. The method of claim 70, wherein the NK antagonist is a NK3 antagonist.

72. The method of claim 71, wherein the NK3 antagonist is osanetant, or a pharmaceutically acceptable salt thereof.

73. The method of claim 70, wherein the patient undergoing bilateral salpingo-oophorectomy is a breast cancer patient.

74. The method of any one of claims 70-73, wherein the NK antagonist is administered twice a day, and the total daily dose of the NK antagonist ranges from about 50 mg per day to about 200 mg per day.

75. The method of any one of claims 70-74, wherein the NK antagonist is administered prior to the bilateral salpingo-oophorectomy for a period of less than one week.

76. The method of any one of claims 43-75, wherein the patient continues to receive a NK antagonist after the hormone deprivation therapy, a medical and/or surgical procedure.

77. The method of any one of claims 43-75, wherein the NK antagonist is administered concurrently with hormone deprivation therapy, a medical and/or surgical procedure.

78. The method of any one of claims 1-77, wherein the method alleviates social isolation stress in the cancer patient.
